(19) United States
(12) Patent Application Publication
(10) Pub. No.: US 2020/0317764 A1
(43) Pub. Date: Oct. 8, 2020

(54) MODIFIED MRNA FOR MULTICELL TRANSFORMATION

(71) Applicant: MORPHOGENESIS, INC., Tampa, FL (US)

(72) Inventors: MICHAEL J. P. LAWMAN, TEMPLE TERRACE, FL (US); PATRICIA D. LAWMAN, TEMPLE TERRACE, FL (US); MEGHAN GENTILINI, ST. PETERSBURG, FL (US); VIJAY RAMIYA, TAMPA, FL (US); MARINA VICTOR ABDELMASEEH BASTAWROUS, PORT ORANGE, FL (US); MICHAEL J. SHAMBLOTT, ST. PETERSBURG, FL (US)

(21) Appl. No.: 16/869,642

(22) Filed: May 8, 2020

Related U.S. Application Data
(63) Continuation-in-part of application No. 15/583,599, filed on May 1, 2017, now Pat. No. 10,682,401.
Application No. 15/583,599 is a continuation-in-part of application No. 15/114,943, filed on Jul. 28, 2016, now Pat. No. 9,636,388.
(60) Provisional application No. 62/163,446, filed on May 19, 2015.

(51) Int. Cl. C07K 16/18; C12N 15/85; C12N 15/70; A61K 39/00; A61P 35/00; A61K 48/00

(52) U.S. Cl. CPC C07K 16/18; C12N 15/85; C12N 15/70; A61K 39/0011; A61K 2039/53; A61K 48/00; A61K 2039/5152; A61K 2039/5156; A61K 2039/552; A61P 35/00

(57) ABSTRACT

Synthetic bacterial messenger RNA can be used to prepare autologous, allogenic or direct nucleic acid cancer vaccines. Cancer cells are transfected either in vitro or in vivo with mRNA obtained from DNA that encodes an immunogenic bacterial protein. An immune response to the cancer is generated from direct administration of the mRNA in vivo or administration of vaccines prepared from cancer cells in vitro. Codon modification of the mRNA can optimize expression of an immunogenic polypeptide in cancer cells.

### CROSS-REFERENCE TO RELATED APPLICATIONS

This application is a continuation-in-part of U.S. application Ser. No. 15/583,599, filed May 1, 2017, which is a continuation-in-part of U.S. application Ser. No. 15/114,943, filed Jul. 28, 2016, now U.S. Pat. No. 9,636,388, which is the U.S. national stage application of International Patent Application No. PCT/US2016/033235, filed May 19, 2016, which claims the benefit of U.S. Provisional Application Ser. No. 62/163,446, filed May 19, 2015, the disclosures of which are hereby incorporated by reference in their entirety, including all figures, tables and amino acid and nucleic acid sequences.

The Sequence Listing for this application is labeled “Seq-List.txt” which was created on Apr. 12, 2020 and is 31 KB. The entire content of the sequence listing is incorporated herein by reference in its entirety.

### BACKGROUND OF THE INVENTION

### 1. Field of the Invention

The invention relates generally to vaccines and particularly to cancer vaccines prepared by either transfection of cancer cells or direct intratumoral administration with a synthetic bacterial messenger ribonucleic acid (mRNA).

The invention more particularly describes synthetic RNAs that efficiently express a selected polypeptide in mammalian cells and the use of the RNAs to transform the cells in vivo or in vitro.

The present invention provides for development and use of effective mRNA vaccines for cancer treatment. While deoxyribonucleic acid (DNA) vaccines have several deficiencies, including low transfection efficiency and time consuming delivery methods, the mRNA vaccines of the present invention are administered directly into tumor cells and immediately translated into an immunogenic protein which evokes a multi-tumor-antigen response. The short in vivo half-life of mRNA makes it less likely to integrate into the host genome compared to plasmid DNA and is therefore considered safer. Unlike DNA, mRNA-vaccines do not need to cross the nuclear envelope and thus can often generate more rapid and higher levels of protein expression. Additionally, expression of transfected mRNA is cell cycle independent and since the level of mRNA is not driven by a promoter, protein expression and vaccine dosage can be modulated by changing the level of mRNA transfected. In contrast to peptides, mRNA vaccines lack Major Histocompatibility Complex (MHC) haplotype restriction and can be designed to be self-adjuvating with the addition of MHC I trafficking signal or by combination with protamine. Although the efficacy of mRNA vaccines may benefit from complexing agents which protect RNA from degradation and enhance cellular uptake, cells in vivo can be transfected with mRNA in the absence of other reagents or physical transduction methods. Messenger RNA for use as a vaccine can be generated from plasmid DNA using in vitro transcription.

### 2. Description of Background Art

Treatment for cancers is based on the specific type that is diagnosed. Some common cancers include bladder, breast, colon, lymphoma, melanoma and prostate. Treatment regimens are prepared by physicians based on the evaluation of multiple factors including, but not limited to, disease stage, etiology and patient age and general health. For many cancers the treatment regimen can include one or a combination of surgery, chemotherapy, radiation, bone marrow/stem cell transplants, cancer drugs or immunotherapy. The most common treatments include surgery, chemotherapy, radiation and oral drugs. Although these treatments can be effective there are often many side effects. Chemotherapy in particular targets all newly dividing cells in the body not just the cancerous cells.

The advantage of some immunotherapies is the ability to target the diseased cells while leaving the non-diseased cells intact. Cancerous cells arise from a breakdown in normal growth regulatory mechanisms; therefore, the body still sees many of these cells as self. Cancer immunotherapy overcomes the body's tolerance of these diseased self-cells and allows the body to distinguish them as foreign. Cancer can also escape immune detection through direct suppression of the body's immune system by decreasing expression of immune activating markers on cells such as MHC molecules. The MHC is one of the components that help the body differentiate which cells are self and which are foreign or diseased.

Treatments for solid cancers typically include chemotherapy and/or surgery. Recently there has been interest in developing vaccines in an effort to stimulate an autologous immune defense. U.S. Pat. No. 7,795,020 describes in detail a lymphoma vaccine for treating advanced stages of lymphoma with transformed autologous or non-autologous cells isolated from a subject diagnosed with lymphoma. The isolated cells are transfected with a plasmid vector carrying a Streptococcus pyogenes emm55 gene. The bacterial protein is expressed on the cell surface and when the transfected cells are introduced to a subject with the cancer, generates an immunological response to lymphoma cells.

To date the FDA has approved only cellular cancer immunotherapy vaccine, Provenge, for the treatment of prostate cancer; however, some vaccines are currently being tested in clinical trials. Biovaxld is an autologous tumor derived immunoglobulin idiotype vaccine undergoing Phase III clinical studies in the treatment of indolent follicular Non-Hodgkin Lymphoma.

In principle, either exogenous DNA or RNA can express proteins in the mammalian body. Whether or not similar immune activity can be produced with both DNA and mRNA expressed proteins is uncertain. Conventional wisdom is that DNA is superior for the creation of vaccines and gene therapy due to its stability and ease of use. An example of a plasmid DNA vaccine is Merial's Oncept, which was developed for treatment of oral canine melanoma.

Work on mRNA vaccines has been reported. In one case, an effective mRNA vaccine was delivered using liposomes. This particular vaccine induced cytotoxic T lymphocytes in vivo after administration of mRNA encoding an influenza virus protein into mice. Other studies by CureVac GMH indicated that the mRNA vaccine elicits a humoral and cellular immune response upon delivery intradermally. This vaccine was administered in naked form and also complexed with protamine, a protein that enhances mRNA stability and improved protein expression. This vaccine is currently in clinical trials for castration-resistant prostate cancer.

Human trials have been performed using mRNA on liquid and solid tumors. The cancers include acute myeloid lymphoma, metastatic melanoma, prostate cancer, renal cell carcinoma/ovarian carcinoma, neuroblastoma, brain, lung, colon, and renal cell carcinoma. Most of the clinical trials that are currently being carried out involve the transfection of mRNA into autologous dendritic cells, rather than cancer cells. Additionally, no clinical trials using intratumoral administration of mRNA have been attempted. FIG. 3 is a table of published clinical trials using mRNA vaccines.

Delivery vehicles such as liposomes and cationic polymers appear to have promise in enhancing transfection. Once the liposome or polymer complex enters the cytoplasm, the mRNA must be able to separate from the delivery vehicle to enable antigen translation; unfortunately, these vehicles may not properly complex with mRNA and therefore not allow for proper translation of the encoded protein. Antigen production may occur but in amounts insufficient to produce a desired effect.

Many immunotherapies are disease-specific, complicated in concept and even more complicated and expensive to produce. It remains to be seen whether such therapies will be commercially viable. The administration of mRNA directly into a patient's tumor where it is immediately translated into an immunogenic protein which evokes a multi-tumor-antigen response has far-reaching implications. For instance, a single synthetic mRNA can be used to treat multiple types of cancer in multiple species. mRNA is simple to deliver, cost-effective, easily transported and stored, as well as easy to administer. Along with an excellent safety profile, these attributes of mRNA make it possible to treat cancer patients worldwide, even in developing countries.

Guiding the immune system to kill cancer cells is the basis for all cancer immunotherapies. In order for any type of immunotherapy to succeed, an immune response to tumor associated antigens must be triggered and allowed to amplify. The immune response can involve any number of immune cells including antigen presenting cells, neutrophils, natural killer cells, T helper cells, T cytotoxic cells and B cells, etc. However, the triggering and activation of an immune response to single tumor antigens has not proven adequate to translate into beneficial clinical efficacy in human cancer vaccine trials, most likely due to immune escape variants; nor has using whole tumor cells or tumor cell lysates plus exogenous adjuvants as a supplier of multiple relevant tumor antigens. That is why it is imperative to be able to supply the trigger in the context of the tumor antigens as they are expressed on the patient's tumor cells. The only way to accomplish this is to provide the encoding nucleic acid to the tumor cell so that the cellular machinery can express the trigger antigen alongside the tumor antigens in such a way that all of these antigens are exposed to the cells of the immune system. Such exposure then results in interantigenic epitope spreading so that an adaptive immune response is educated and activated against all tumor cells bearing those antigens, even in the absence of the trigger antigen.

Using nucleic acids as vaccines has multiple other advantages. Nucleic acid vaccines can induce both humoral and cellular immune responses; have low effective dosages; are simple to manipulate; avail rapid testing; are cost-effective and reproducible in large scale production and isolation; can be produced at high frequency and are easily isolated; are more temperature-stable than conventional vaccines; have a long shelf-life; are easy to store and transport; and are unlikely to require a cold chain.

DNA has been used in vaccines with success. DNA is a double stranded molecule that serves as the blueprint, i.e., genetic instructions, for organisms. DNA is amenable to use as a vaccine as it is fairly stable and unreactive and can be stored long term. However, DNA is self-replicating and can be easily damaged by ultra-violet radiation.

On the other hand, RNA is single stranded and functions to carry out the DNA's instructions, i.e., RNA transfers the genetic code to create proteins. RNA is more reactive than DNA and less stable but is resistant to ultra-violet radiation. As it turns out, these latter qualities make RNA better suited to use as vaccines. In general mRNA has zero chance of integrating into the host chromosomes. The delivery of mRNA results in faster expression of the antigen of interest and requires fewer copies for expression. mRNA expression is transient, which seems like a disadvantage but actually adds to its safety. mRNA is more effective than DNA for protein production in post mitotic and non-dividing cells because DNA requires translocation through the nuclear member and plasmid membrane, while mRNA requires translocation only through the plasmid membrane. mRNA is not only a template for translation, but also acts as a ligand for toll-like receptors and is nuclease sensitive; therefore it presents less concern for horizontal transmission.

### SUMMARY OF THE INVENTION

The invention is based on use of a ribonucleic acid message (mRNA) (SEQ ID NO: 1, SEQ ID NO: 13, SEQ ID NO: 15 and SEQ ID NO: 16) that encodes an immunogenic bacterial protein. The message can be delivered into the cell cytoplasm using any of a number of known techniques. Once the mRNA reaches the cytoplasm, it is translated into the encoded protein using the cellular machinery already in place. The bacterial protein, such as an M-like protein having the amino acid sequence of SEQ ID NO: 2 or SEQ ID NO: 14, will then be expressed in the cell rendering immunogenicity to a cancer cell. For example, M-like proteins can be derived from the bacterial sources, Group A and G Streptococci (GAS and GGS), and therefore are seen by the mammalian body as foreign. Immune monitoring cells, such as antigen presenting cells (APCs), are attracted by the foreign protein. The APC's will phagocytize the entire cancer cell and then present all the foreign/mutant proteins including M-like protein to other immune cells.

Production of the bacterial proteins in the cells is achieved by insertion of the corresponding genetic code. The gene for M-like proteins is referred to as emmL. Once the emmL message is delivered into a cancer cell containing abnormal proteins produced by the mutation in the cell's DNA, the M-like protein will be expressed in the cell, attracting immune cells to engulf it, and lead to the presentation of the previously-masked mutated proteins to the immune system. Abnormal proteins may have been present for an extended period of time, but because they were derived from “self” proteins, the body would not necessarily view them as foreign or a threat. The bacterial protein antigen acts as a primer or trigger for the immune system to address cells that it otherwise may not have been able to identify previously as damaged and harmful.

The mRNA is produced as described in the examples. Once obtained, the mRNA containing the immunogenic message can be delivered into autologous or allogeneic cells that require the priming affect described in the summary of invention. The mRNA can also be directly delivered intratumorally or in the case of some cancers such as lymphoma, intranodally as well.

One M-like protein encoded by an emmL gene has previously been delivered into cells through DNA and has been shown to be expressed in the cell to produce an immunological effect. Due to concerns with DNA delivery, including the possibility of gene integration into the chromosomes, delivery of the message via RNA is a safer alternative because it cannot integrate into the host DNA. This ability of DNA to integrate into host DNA becomes especially relevant in medical applications where exogenous DNA integration can create detrimental effects. In contrast to DNA expression, mRNA expression lasts only a few hours to a few days at maximum inside a cell. mRNA that is not delivered into the cells is quickly degraded by RNases that are present in the environment and therefore does not pose the risk of being horizontally transmitted. When an emmL mRNA is successfully transfected into a cancer cell, it can express an immunogenic bacterial protein in the cancer cell and on its surface and thereby induce an immunogenic response.

### BRIEF DESCRIPTION OF THE FIGURES

FIG. 1 illustrates the plasmid used for the backbone of the recombinant plasmid designed for mRNA production of M-like protein.

FIG. 2 illustrates the plasmid DNA used as the source for the emmL gene to be ligated into the linearized vector of FIG. 1.

FIG. 3 is a summary of mRNA trials performed in solid cancers.

FIG. 4 is a diagram demonstrating the cellular production pathway differences between mRNA and DNA.

FIG. 5 shows the creation of a recombinant DNA vector to produce an mRNA encoding a bacterial antigen.

FIG. 6 shows a Western blot of isolated Emm55 against an anti-M-like antibody.

FIG. 7 shows comparative results of protein expression from DNA and RNA transfection.

FIG. 8 is a photograph of an agarose gel of synthesized emm55 mRNA using a Flashgel system.

FIG. 9 is a graph showing antibodies reactive to EmmL protein in mice vaccinated with emmL mRNA or water (controls) (C2) after weeks 1, 2, 3 and 4.

FIG. 10 is a Western blot showing presence of antibodies in mice blood that react to EmmL protein pre and post vaccination with emmL mRNA.

FIG. 11 is the map of a novel double stranded DNA molecule used in the production of synthetic mRNA that can be translated at high efficiency in mammalian cells. This DNA molecule contains the sequences for T7 RNA polymerase (SEQ ID NO: 8), a portion of the Xenopus laevis beta globin gene 5′ untranslated region (SEQ ID NO: 9), a polylinker with restriction endonuclease recognition sites for SacI, NotI, BglII, EcoRV and SpeI used to insert the coding region of emmL (SEQ ID NO: 7), a portion of the Xenopus laevis beta globin gene 3′ untranslated region (SEQ ID NO: 10), then a polylinker with restriction endonuclease recognition sites for BamHI, EcoRI and XbaI used to linearize the plasmid prior to in vitro transcription (SEQ ID NO: 11). The DNA sequence corresponding to FIG. 11 is SEQ ID NO: 12.

FIGS. 12A-12B show relative adaptiveness plot of emm55. Y axis is the adaptiveness index for each codon of emm55, where 1.0 is perfectly adapted for human cell expression.

FIG. 12A is the original emm55 sequence.

FIG. 12B shows emm55 after optimization with the JCat algorithm.

FIGS. 13A-13C are simulated western blots from Wes™ capillary electrophoresis analysis of Emm55 expressed following transient transfection of HEK293T and B16-F10 cells with wild type uridine (WT) and N1-methyl pseudouridine containing mRNA. Lysates were at 0.5 μg/μL. ERK1 used as a normalizing control. −, cells transfected with EGFP mRNA. *, 6 hr post Emm55FreqDist transfection HEK293T cell lysate incubated with rabbit anti-ERK1 only.

FIG. 13A is emm55Jcat.

FIG. 13B is emm55MostFreq.

FIG. 13C is emm55FreqDist. *, 6 hr post Emm55FreqDist transfection HEK293T cell lysate incubated with rabbit anti-ERK1 only.

FIGS. 14A-14B show the normalized expression of Emm55 from images shown in FIG. 13. Codon Adaptiveness Index is used to determine codon optimization in the JCat algorithm, where 1.0 is perfectly optimized.

FIG. 14A shows expression in HEK293T cells.

FIG. 14B shows expression in B16-F10 cells.

FIGS. 15A-15B show the time course of Emm55 expression in HEK293T cells.

FIG. 15A is the initial assay performed at the Wes™ maximum signal intensity.

FIG. 15B is a replica experiment with reduced protein load. Significance determined by One-way ANOVA with Bonferroni post-hoc testing. *, p<0.05; **, p<0.01, ***, p<0.001, ****, P<0.0001 (n=3). Open star, significance between emm55JCat-N1 and pAc/emm55. Closed star, significance between emm55JCat-WT and pAc/emm55.

### DETAILED DESCRIPTION OF THE INVENTION

The present invention provides a cancer vaccine that can be prepared efficiently and with less expense than previously used vaccines, which are prepared by introducing plasmid DNA directly into the nucleus of the cell. The use of an emmL encoding mRNA (SEQ ID NO: 1, SEQ ID NO: 13, SEQ ID NO: 15 and SEQ ID NO: 16) inserted into the cell cytoplasm is more effective in transfecting tumor cells.

The present invention further provides several new emmL encoding RNAs designed to optimize increased expression in transformed cells. The more robust expression of Emm55 protein was achieved using several codon optimization algorithms designed to adapt the emm55 nucleotide sequence to mammalian expression.

mRNA is capable of antigenic protein message delivery into cancer cells. Not only is mRNA a safer alternative because it cannot integrate into the host DNA, but also expression is limited to only a few hours to a few days at maximum. mRNA delivery also places the antigenic message further down the cellular protein production process, therefore providing a quicker expression in the cells. The mRNA only has to be delivered into the cell cytoplasm, whereas DNA must ultimately end up in the nucleus to be effective. The use of mRNA is an advantage for production of protein antigen because mRNA can induce protein production in both post mitotic and non-dividing cells.

Although mRNA delivery of antigenic proteins is a safer alternative to DNA delivery, stability and immunogenicity of mRNA must be addressed. Many of the elements that facilitate increased stability and immunogenicity were engineered into the recombinant vector template. If appropriate elements are not contained in the vector, they can be added; for example, if the template vector does not contain a poly(A) tail coding sequence, the tail is added during transcription.

In order to have increased stability in the cytoplasm, the mRNA must contain both a 5′-methylguanosine cap and a 3′-poly(A) tail (SEQ ID NO: 3). These elements are responsible for attracting and attaching the components of the cell machinery responsible for translating the mRNA into proteins. Absence of these components can decrease the time in which the mRNA is available for protein translation prior to degradation. Accordingly, these elements have been incorporated into the mRNA as described in the examples.

Efficient immunogenicity can be increased by utilization of techniques such as enhanced delivery with viral vectors, nanoparticles, cationic polymers, lipids and electroporation. Viral vectors have been used extensively in delivery of plasmid DNA (pDNA) but have several risks, as well as increased cost. Electroporation with mRNA is less toxic to cells due to less stringent electrical settings and is a preferable method for delivery of mRNA. DNA requires a higher electrical charge to pass the DNA through the outer cellular membrane and the nuclear membrane, while mRNA needs to pass only though the outer cellular membrane.

Production of mRNA for vaccines has both economic and production benefits compared to pDNA. mRNA is synthesized in vitro from a linearized pDNA template and only a small amount of DNA is required. On the other hand, production of large amounts of pDNA is labor intensive and requires equipment such as large fermentation tanks to grow sufficient bacteria to produce the massive amount of pDNA required for vaccine production. While pDNA isolated from large cultures is pure, due to the circular nature of plasmids the end product occurs in three structural forms; relaxed, linear and supercoiled. Although each form has the ability to produce an antigenic protein once inside a cell, each DNA form varies in its ability to enter the cell via the plasma membrane. Production of mRNA creates only one structural form. Moreover, due to the synthetic method of production the batch to batch reproducibility is high.

From a production standpoint, mRNA is synthesized from DNA and is highly reproducible. This is important for use as a vaccine because no large scale growth is required, i.e. it takes less time and materials and there is less risk of contamination. These factors contribute to reduced costs. Further, synthesis of mRNA leads to higher yield since it only takes one linearized plasmid DNA to yield one hundred mRNA molecules. mRNA is produced in vitro, so there is no E. coli contamination post isolation (genomic DNA or endotoxin). This leads to fewer purification steps and quality control tests. The synthetic nature of in vitro transcription also ensures better batch to batch reproducibility and purer product since vector sequences, including selection markers, are not part of final product. Also in contrast to DNA, mRNA has a single molecular conformation, whereas plasmid DNA has three. mRNA is also easier to transfect than plasmid DNA and results in less cell death during electroporation since lower voltage is required. Like DNA, mRNA can also be lyophilized. From a regulatory standpoint, mRNA is safer because it is non-replicating and is transient. mRNA also poses minimal to no environmental issues since it is easily degraded and confers no antibiotic resistance.

The below comparison illustrates the advantages of using mRNA instead of DNA for antigenic emmL message delivery into cancer cells. The comparison is broken up into three parts; upstream production, downstream production and cellular delivery. The bulk of the benefits, including decreased production cost, reduced manufacturing time, superior message delivery and increased safety, are seen in the upstream production and cellular delivery. Each section shows a large difference between the DNA and mRNA processes as well as similar steps for each process.

Upstream Production:

The upstream production of both nucleic acid products is almost identical up to bacterial culture expansion. Only a small amount of DNA is required to produce approximately 100 times the amount of mRNA. For example, in vitro transcription experiments have yielded 25 μg of mRNA from only 0.2 μg of DNA. This is 25 times more mRNA than DNA produced using the same amount of culturing. Culture expansion can be very expensive and time consuming and leads to increase risk of contamination or mutation of DNA.

The benefit of having to grow only a small bacterial culture is significant. The small amount of DNA from this culture requires a smaller isolation to be performed. This downsizing saves time and resources and decreases contamination risk. The production of the final mRNA product requires an additional step of transcribing the mRNA from the DNA template. This is a synthetic step performed in vitro. Due to the synthetic nature of transcription, there is good batch to batch reproducibility and the procedure takes only a few hours. Culturing DNA-containing bacteria can require up to several days.

A significant disadvantage of using pDNA rather than mRNA is that the end product has the potential to be contaminated with genomic DNA (gDNA). Also, the isolated pDNA forms three configurations; linear, super-coiled and circular that do not transfect cells with the same efficiency. The mRNA final product is pure, in a single conformation, and is not contaminated with gDNA or pDNA.

Chart 1 compares steps employed for DNA and mRNA production.

CHART 1

| Process Steps | DNA | Process Steps | mRNA |
|---|---|---|---|
| Vector | Designed to maximize | Vector | Designed to create a |
| Engineering | number of plasmid DNA | Engineering | stable mRNA molecule |
| | (pDNA) copies created in | | that will lead to |
| | each bacterium | | increased protein |
| | | | expression |
| Transformation | Vector transformed into | Transformation | Vector transformed |
| of Bacteria | competent E. coli | of Bacteria | into competent E. coli |
| Growth | Transformed E. coli used to | Growth | Transformed E. coli |
| Bacterial | inoculate a small culture for | Bacterial | used to inoculate a |
| Culture | further expansion | Culture | small culture for later |
| | | | harvesting and |
| | | | purification |
| Culture | Culture must be expanded | N/A | Culture expansion not |
| Expansion | to 2.5 L-1000 L depending | | needed for mRNA |
| | on how much DNA is | | because only a small |
| | needed | | quantity of DNA is |
| | Depending on the desired | | needed to synthesize |
| | quantity this step can add | | mRNA |
| | days or weeks to the process | | Approx. one linearized |
| | | | pDNA = 100 mRNA |
| | | | molecules |
| Harvesting | Large scale must be | Harvesting | Small scale due to |
| | performed to generate | | above |
| | adequate amount of DNA | | Saves time |
| | Labor intensive | | |
| Plasmid | End product can have a | Plasmid | Small scale due to |
| Isolation and | genomic DNA | Isolation and | above |
| Purification | contamination | Purification | Saves time |
| | 3 conformation of DNA | | |
| | produced, not all transfect | | |
| | efficiently | | |
| N/A | End product is purified | mRNA | RNA is synthesized |
| | plasmid DNA | Transcription | from linearized pDNA |
| | | and Purification | Once synthesized it |
| | | | must be purified |
| | | | Good batch to batch |
| | | | reproducibility |

Downstream Production (Autologous Preparation):

The majority of downstream production is the same for DNA and mRNA. One difference lies within the electroporation step. mRNA requires a lower voltage since it only has to pass through the plasma membrane and not the nuclear membrane, unlike DNA, which must pass through both the plasma and nuclear membranes. A lower voltage is favorable because it results in less cell death during electroporation. The increased viability of the mRNA transfected cells translates into an adequate proportion of vaccine cells expressing M-like proteins with ease.

Chart 2 compares processing of DNA and mRNA in tumor tissue through preparation to vaccination in transfected cells.

CHART 2

| Process Steps | DNA | Process Steps | mRNA |
|---|---|---|---|
| Specimen | Tumor tissue is excised | Specimen | Tumor tissue is excised |
| Arrival and | from the patient and | Arrival and | from the patient and |
| Processing | shipped to the | Processing | shipped to the laboratory |
| | laboratory | | If the tumor tissue is solid, |
| | If the tumor tissue is | | it is digested using |
| | solid, it is digested | | enzymes to release cells. If |
| | using enzymes to | | from lymphoma, cells are |
| | release cells. If from | | aspirated |
| | lymphoma, cells are | | |
| | aspirated. | | |
| Cell Cultivation | The released cells are | Cell | The released cells are |
| | cultured to expand total | Cultivation | cultured to expand total cell |
| | cell number, if | | number, if necessary |
| | necessary | | If the cell number is |
| | If the cell number is | | adequate, can proceed to |
| | adequate, can proceed | | transfection |
| | to transfection | | |
| Transfection | Must have enough | Transfection | Less voltage needed |
| | voltage to pass through | | because only need to pass |
| | two membranes; | | through the plasma |
| | plasma and nuclear | | membrane |
| Irradiation | Transfected cells are | Irradiation | Transfected cells are |
| | irradiated so they | | irradiated so they cannot |
| | cannot divide once they | | divide once they are |
| | are administered back | | administered back into the |
| | into the patient | | patient |
| Administration | The vaccine of | Administration | The vaccine of irradiated |
| | irradiated cells is | | cells is administered |
| | administered | | intradermally |
| | intradermally | | |

Cellular Delivery:

Significant advantages of using mRNA delivery are demonstrated in the cellular delivery flow chart below. As shown in the chart, mRNA delivery into the cells skips ahead to immediate translation into the antigenic M-like protein. Not only does transfected DNA have to pass through an additional cellular membrane, but it also has to be transcribed into mRNA for delivery back into the cytosol, which is the starting point for the protein synthesis initiated by the mRNA vaccine.

mRNA vaccines can be conjugated with compatible immunologic adjuvants or repressors depending on the effect desired. Adjuvants such as TriMix, a cocktail of immunostimulatory molecules, can be added to an mRNA-based vaccine eliciting an increased immune response against the encoded immunogen. Immunologic repressors can be useful to combat immunosuppressive enzymes of other elements that may hinder the body's ability to mount a sufficient immune response. These immunosuppressive elements can be silenced by using silencing RNA (siRNA) that can be co-delivered during immunization. An additional type of immune repressor that can be administered in conjunction with an mRNA based cancer vaccine is a check-point inhibitor. These generally consist of antibodies, such as anti-PD1 and anti-CTLA4, that bind to receptors present on tumor cells or immune activated cells that if left unblocked will induce immune suppression. This process has been termed as “taking off the brakes” and as it implies this release of the “brakes” allows an immunotherapy, such as the mRNA cancer vaccine, to hone the immune system efforts on attacking the cancerous cells.

The vaccine can be used not only in conjunction with checkpoint inhibitor therapy but also chemotherapy, radiation therapy, whole cell vaccines, other nucleic acid therapy, natural killer cell therapy or chimeric antigen receptor therapy prior to or concurrently with administration of the RNA vaccine.

In other cases, a cancer patient is treated with regimens that alter the tumor microenvironment, including but not limited to, cytokines, anti-fugetaxis agents, chemotactic agents and metronomic doses of chemicals prior to or concurrently with administration of the vaccine.

Chart 3 compares DNA and mRNA processing in cells from cell entry to translation.

CHART 3

| Process Steps | DNA | Process Steps | mRNA |
|---|---|---|---|
| Enter Plasma | DNA must first pass | Enter Plasma | mRNA only has to |
| Membrane | through the plasma | Membrane | enter the cytosol to |
| | membrane | | become active |
| Enter Nuclear | Next the DNA must | N/A | The cellular |
| Membrane | pass through the | | machinery needed to |
| | nuclear membrane | | process the mRNA is |
| | | | located outside the |
| | | | nucleus so this step is |
| | | | not required |
| | | | Leads to quicker |
| | | | protein expression |
| Transcription into | Once in the nucleus the | N/A | The step of |
| mRNA | DNA will be | | transaction has |
| | transcribed into mRNA | | already been |
| | | | accomplished |
| | | | previously in vitro |
| | | | during the upstream |
| | | | production |
| Exit Nucleus | After the mRNA | N/A | The mRNA never |
| | message has been | | enters the nucleus so |
| | created it needs to pass | | this step does not |
| | back through the | | apply to mRNA |
| | nuclear membrane to | | |
| | reach the cytosol | | |
| mRNA | The mRNA is | mRNA | The mRNA is |
| Translation | translated as soon as it | Translation | translated as soon as |
| | reaches the cytosol | | it reaches the cytosol |

In examples 23-25, mRNA encoding emmL can be produced using an in vitro transcription reaction. Several modifications to the resultant mRNA can be made in this reaction to improve mRNA and EmmL protein stability and translation efficiency, and to reduce mRNA immunogenicity. For example, a modified nucleic acid can be attached to the 5′ end of emmL mRNA such as, but not limited to, anti-reverse Cap Analog [ARCA, P1-(5′-(3′-O-methyl)-7-methyl-guanosyl) P3-(5′-(guanosyl))triphosphate)], N1-methyl-guanosine, 2′ fluoro-guanosine, 7-deaza-guanosine, inosine, 8-oxo-guanosine, 2-amino-guanosine, LNA-guanosine and 2-azido-guanosine.

In another example, a poly(A) tail approximately 50-200 adenosine monophosphates in length can be attached to the 3′ end of emmL mRNA or both 5′ modified nucleotide cap and poly(A) tail can be added to emmL mRNA.

emmL mRNA can be synthesized with ribonucleotide analogs. Chemical modifications can be made at this stage to further improve translation efficiency and stability. Examples include; 5-methyl-cytidine-5′-triphosphate, pseudouridine-5′-triphosphate, 2-thiouridine-5′triphosphate, and N1-methylpseudouridine-5′-triphosphate.

There are numerous methods to deliver mRNA to a cell such that EmmL protein will be produced at high levels. For example, emmL mRNA produced following in vitro transcription can be injected directly into a tissue or tumor.

Complexing agents such as lipids or polymers can be used to protect RNA from degradation, enhance uptake by cells and improve delivery to the translation machinery in the cytoplasm. In one embodiment, emmL mRNA is complexed with a liposome prepared from a lipophilic material such as cholesterol and synthetic phospholipids.

In one embodiment, emmL mRNA is complexed with a liposome prepared from a lipophilic material such as cholesterol and natural phospholipids.

In one embodiment, emmL mRNA is complexed with a cationic lipid such as, but not limited to, N-[1-(2,3-Dioleoyloxy)propyl]-N,N,N-trimethylammonium methyl-sulfate (DOTAP).

In one embodiment, emmL mRNA is complexed with a zwitterionic lipid such as, but not limited to, 3-[(3-Cholamidopropyl)dimethylammonio]-1-Propanesulfonate (CHAPS).

In one embodiment, emmL mRNA is complexed with a PEGylated lipid such as, but not limited to, N-(Carbonyl-methoxypolyethyleneglycol 2000)-1,2-distearoyl-sn-glycero-3 phosphoethanolamine (DSPE-PEG).

In one embodiment, emmL mRNA is complexed with a mixture of cationic, zwitterionic and PEGylated lipids.

In one embodiment, emmL mRNA is complexed with protamine.

In one embodiment, emmL mRNA is complexed with a liposome prepared from a specific material such as the Hemagglutinating virus of Japan, which has been used to prepare mRNA vaccine for the treatment of melanoma [26].

In one embodiment, emmL mRNA can be complexed with a polymer that is rationally designed with multiple materials that mimic viral components to transfect specific cells with high efficiency. These would include, but are not limited to membrane-disrupting peptides, nucleic acid binding components, a protective coat layer, and an outer targeting ligand.

In one embodiment, complexing components can be combined and formulated into a single nanoparticle.

In one embodiment, emmL mRNA or emmL mRNA complexes can be combined with interfering RNAs or interfering RNA complexes directed against immune checkpoint molecules such as programmed cell death protein 1 (PD-1), programmed death-ligand 1 (PD-L1), cytotoxic T-lymphocyte associated protein 4 (CTLA-4) or T-cell immunoreceptor with Ig and ITIM domains (TIGIT). One non-limiting example is emmL mRNA complexes and short interfering RNA (siRNA) complexes targeted to PD-1.

In one embodiment, the emmL coding region can be combined with viral RNA replication genes to form a linear RNA molecule capable of self-replication. This linear RNA molecule can then be formulated with lipophilic compounds and lipids to form liposomes capable of transfecting cells with self-replicating mRNA

In one embodiment, emmL mRNA or emmL mRNA formulated with lipids, protamine or in liposomes, is complexed with a biodegradable polymer. One non-limiting example is polycaprolactone, which has been approved by the Food and Drug Administration for use as a drug delivery device. The advantage of biodegradable polymer complexing is that mRNA or mRNA complexes can be delivered over a long-time frame as the polymer degrades. This sustained delivery can enhance the effectiveness of a vaccine.

In one embodiment, emmL mRNA or emmL mRNA complexes can be formulated in a biodegradable polymer containing tissue- or tumor-specific factors that enhance the efficacy of the emmL mRNA vaccine. An example can be an emmL mRNA vaccine formulated with a biopolymer containing factors that inhibit angiogenesis or vasculogenesis, thereby providing a synergistic anti-tumor effect.

In one embodiment, emmL mRNA or emmL mRNA complexes can be formulated in a biodegradable polymer containing factors that reduce the expression of immune checkpoint molecules such as PD-1, PD-L1, CTLA-4 or TIGIT. As a non-limiting example, this can include existing or novel pharmaceutical reagents such as small molecules or antibodies that reduce the expression of PD-1, thereby providing a synergistic anti-tumor effect.

In one embodiment, emmL mRNA or emmL mRNA complexes can be formulated in a biodegradable polymer containing interfering RNAs or interfering RNA complexes for factors that reduce the expression of immune checkpoint molecules such as PD-1, PD-L1, CTLA-4 or TIGIT. One non-limiting example is emmL mRNA complexes and short interfering RNA (siRNA) complexes.

In one embodiment, emmL mRNA or emmL mRNA complexes can be combined with an adjuvant such as synthetic double-stranded RNA polyriboinosinic polyribocytidylic acid [poly(I:C)].

Analysis of pSFCMVT/emmL Design Elements

Several design elements could improve the level of EmmL protein production.

The mRNA vaccine detailed in example 1 (SEQ ID NOs: 1-3) was cloned into the Oxford Genetics pSF-CMV_T7 plasmid DNA vector to produce pSFCMVT7/emmL. Although mRNA expression following transfection of mammalian cells was detected, only low levels of Emm55 protein were detected on western blots. A retrospective analysis of pSFCMVT7/emmL identified design elements that could improve translation efficiency in mammalian cells.

There are at least two different types of design element that could improve the level of EmmL protein production. Proximal elements are close to the emmL coding region and are added during polymerase chain reaction (PCR) amplification of emmL from pAc/emm55. Distal elements are distant from the emmL coding region and are of general utility for enhancing expression of protein from an mRNA.

Proximal elements include the introduction of a translation initiation sequence optimized for mammalian cell expression to the N-terminal end of the emmL coding region. This sequence has been determined to be RYMRMVATGGC, where R is A or G, Y is C or T, M is A or C and V is A, C or G (SEQ ID NO: 4). In one embodiment, the optimal translation initiation sequence, ATAGCCATGGC (SEQ ID NO: 5), replaces the native start codon of emmL.

In one embodiment of this proximal design element, oligonucleotide PCR primers are synthesized such that there is an equimolar concentration of each nucleotide at the degenerate positions (R, Y, M and V in SEQ ID NO: 4) and the final oligonucleotide synthesis product contains all possible primer sequences. In the current embodiment, the optimal translation initiation sequence is determined empirically by comparing the level of EmmL protein expression in assays such as western blots after the emmL gene has been sub-cloned into a mammalian expression plasmid DNA vector.

Another proximal design element that can be used to modify the C-terminal end of the emmL coding region is the addition of stop codons added after the native stop codon. Many eukaryotic expression plasmid vectors contain the DNA sequence of all three stop codon variants, TAG, TAA and TGA, immediately after the last codon of the expressed gene. Since the DNA sequence of the native emmL stop codon is TAG, SEQ ID NO: 6. can be included as an added proximal design element.

Another proximal design element is the addition of restriction endonuclease recognition sites that can be added to the N- and C-terminal ends of the emmL coding region to facilitate its insertion into plasmid cloning vectors. The choice of restriction endonuclease recognition site is made based on the destination plasmid. Some examples of restriction endonucleases that could be used are SacI, NotI, BglII, EcoRV and SpeI. In one preferred embodiment, SacI and SpeI sites are added to the N- and C-terminal ends of the emmL coding region, as they do not cut within the emmL coding region and share reaction conditions, so they can be used simultaneously to prepare the emmL coding region PCR amplimer for insertion into the destination plasmid.

Design elements that are distal to the emmL coding region are added to a plasmid cloning vector such that any variation of the emmL coding region, or other gene that is adapted with the proximal design elements, can be inserted. These distal elements can be engineered to create an mRNA expression vector capable of driving high-level mRNA and protein expression of emmL or any other mammalian mRNA.

One distal design element is the DNA sequence for a bacteriophage RNA polymerase promoter region. Examples of bacteriophage RNA polymerases are T7, T3 and SP6. In one embodiment, the promoter for T7 RNA polymerase (SEQ ID NO: 8) is the first of several distal elements added in such a way that it is ultimately upstream, with respect to the action of T7 RNA polymerase, from the emmL coding region.

RNA sequences immediately 5′ and 3′ of eukaryotic gene coding regions that are transcribed but not translated, termed untranslated regions (UTRs), can have strong positive or negative effects on protein translation from in vitro transcribed mRNA. In general, UTRs that support high level protein expression are unstructured, lack negative regulatory sequences and may contain the binding sites for microRNAs that serve to further refine the gene expression pattern. Both UTRs of genes that are highly expressed in mammalian cells in general, or UTRs from genes with an expression pattern matching the tissue where mRNA vaccine expression is desired, can be used to optimize delivery of a gene product such as EmmL protein. UTRs from the Xenopus laevis beta globin gene have been used extensively to mediate high levels of translation in a wide range of eukaryotic cells, including in the design of RNAs used for mammalian immune therapy Examples of tissue-specific UTRs are the tryptophan hydroxylase (TPH) isoforms, which drive differential expression in the pineal gland and brain stem.

In one embodiment, the Xenopus laevis beta globin gene 5′ UTR can be positioned upstream, with respect to RNA polymerase activity, from the emmL coding region. SEQ ID NO: 9 is one non-limiting example of a Xenopus laevis beta globin gene 5′ untranslated region.

In one embodiment, the Xenopus laevis beta globin gene 3′ UTR can be positioned downstream, with respect to RNA polymerase activity, from the emmL coding region. SEQ ID NO: 10 is one non-limiting example of a Xenopus laevis beta globin gene 3′ untranslated region.

In one embodiment, both the Xenopus laevis beta globin gene 5′ and 3′ UTRs can be positioned to flank the emmL coding region.

In one embodiment, 5′ and 3′ UTRs are selected based on the type of neoplastic cell targeted by the vaccine. In one non-limiting set of examples, the 5′ and 3′ UTRs of genes expressed highly in melanoma cells, such as tyrosinase (TYR), melanogenesis associated transcription factor (MITF), melanocortin receptor 1(MC1R), telomerase (TERT), cyclooxygenase 2 (COX2), C-X-C motif chemokine receptor 4 (CXCR4) and baculoviral IAP repeat containing 5 genes (BIRC5), can be used to provide high levels of cell-specific expression to an emmL-based vaccine when used to treat melanoma.

In one embodiment, other genetic elements not located within the primary gene transcript that confer desired expression level and specificity can be included before, after or within the 5′ and 3′ UTRs to further refine vaccine expression.

Another distal design element is a synthetic DNA sequence that provides restriction endonuclease recognition sites that can be used to insert the emmL coding region into the other flanking distal design elements. In one embodiment, the restriction endonuclease recognition sites for SacI, NotI, BglII, EcoRV and SpeI are used (SEQ ID NO: 7).

Another distal design element is a synthetic DNA sequence that provides restriction endonuclease recognition sites to cut the destination plasmid immediately after the emmL coding region to produce a linear plasmid DNA molecule. Linearization provides effective termination for RNA polymerase activity, which increases the production of uniform length mRNA transcripts. An example of a sequence is SEQ ID NO: 11, which contains restriction endonuclease recognition sites for BamHI, EcoRI and XbaI. In one preferred embodiment, BamHI can be used to linearize a plasmid containing the emmL coding region as it does not cut within the emmL coding region and leaves a 5′ nucleotide overhang, which may allow for higher efficiency transcription than a 3′ nucleotide overhang.

In one embodiment, a double stranded DNA molecule containing the abovementioned distal design elements is synthesized by use of overlapping complementary synthetic single stranded oligonucleotide molecules. These molecules are annealed and gaps remaining are filled in with a DNA polymerase such as Taq polymerase.

In one embodiment, two complementary synthetic single stranded oligonucleotide molecules can be synthesized to encompass all desired distal design elements. These oligonucleotide molecules are then annealed. In either of the abovementioned embodiment, the resultant blunt-ended synthetic DNA molecule can be inserted into a PCR cloning vector such as the Invitrogen pCR II-TOPO plasmid by adding a template non-specific adenosine. This is accomplished by incubating the blunt-ended synthetic DNA molecule with Taq polymerase and deoxyadenosine triphosphate at approximately 200 μM final concentration at 72° C. for 10 minutes.

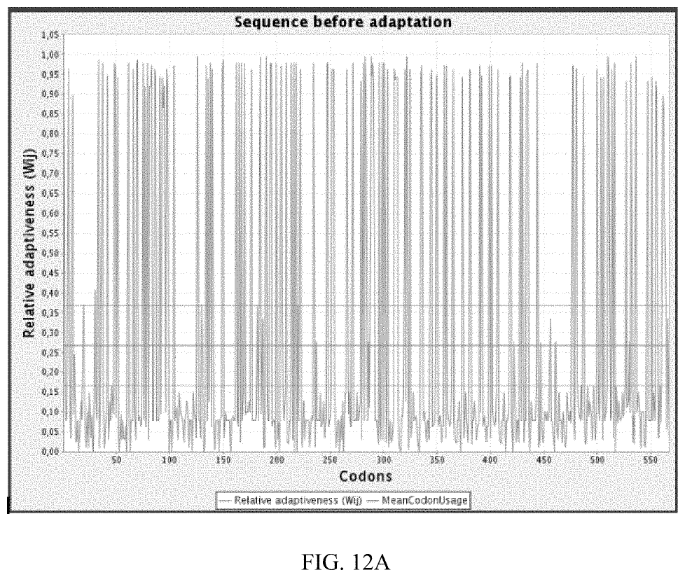

In one embodiment, a plasmid containing the abovementioned distal design elements is named pT7XLUTR. A map of the synthetic DNA molecule inserted into pCR®II-TOPO is shown as FIG. 11 and the nucleotide sequence is shown as SEQ ID NO: 12. This sequence contains the abovementioned distal design elements, but does not represent the totality or full extent of elements that could be added to support the desired expression level or specificity.

pT7XLUTR can be used to transform E. coli. Bacterial transformed with pT7XLUTR can be selected on an agar plates containing an antibiotic that matches the PCR cloning vector, such as agar plates containing Luria Bertani broth supplemented with 50-100 μg/mL kanamycin or carbenicillin. The bacterial culture can be expanded by growth in a liquid antibiotic selective media, such as Luria Bertani broth supplemented with 50-100 μg/mL kanamycin or carbenicillin, and plasmid DNA prepared using methods known to a person practiced in the art.

RNAs incorporating several of the described design elements were found to increase expression of Emm55. The sequences are shown in SEQ ID NO: 13, SEQ ID NO: 15 and SEQ ID NO: 16.

### Examples

The following examples are provided as illustrations of the invention and are in no way to be considered limiting.

### Example 1. Autologous mRNA Vaccine for Canine Lymphoma

A 75 lb. male neutered Rhodesian Ridgeback, presents to his veterinarian with swollen mandibular and inguinal lymph nodes. The patient has a fine needle aspirate performed on one of the enlarged nodes. Upon review by the pathologist he is diagnosed with low grade diffuse lymphoma.

The patient's owner elects to pursue immunotherapy treatment instead of chemotherapy and steroids, due to the minimal side effects reported in immunotherapy treatments. The veterinarian excises the right mandibular lymph node while the patient is under general anesthesia. The tissue sample is shipped overnight for laboratory processing.

Upon receiving the tissue sample at the lab the following is performed: 1) the travel medium is checked for any bacterial contamination, 2) the tissue dimensions are measured, 3) the intact lymph node is aspirated repeatedly using several boluses of wash medium to release the tumor cells, and 4) the aspirated cells are collected and counted.

An appropriate amount of cells is made available to electroporate with emmL encoding mRNA. Using a BioRad Gene Pulse machine, 120×10⁶ cells are transfected with 80 μg of mRNA. A small portion of the transfected cells are cryopreserved, while the rest are placed in culture for approximately 24 hours. After 24 hours the cells are irradiated and aliquoted into 10×10⁶ cell vaccine doses that are cryopreserved until needed.

The patient is administered a total of 8 vaccine doses. Each dose is shipped overnight from the laboratory to the veterinary clinic, arriving on the scheduled administration day. The veterinarian administers each dose intradermally using a syringe with needle. The 8 vaccine doses are given every 7 days (+/−1 day) for 4 weeks and then once a month for 4 months. Prior to the first dose a blood sample is taken. Subsequent blood samples are taken preceding the 5^{th} vaccine, 8^{th} vaccine and 8 weeks after the last vaccine. The blood samples are processed for peripheral blood & plasma and preserved at the lab. They are later used for evaluation of anti-tumor immune response.

Throughout the course of treatment, the patient's lymph node size is monitored along with his overall quality of life. Overall disease state is assessed by tumor burden reduction and anti-tumor immune response. Tumor burden is evaluated through measurements performed on each of the lymph nodes throughout the course of treatment. Anti-tumor immune response is measured using standard enzyme-linked immunosorbent assay (ELISA) to assess antibody levels and flow cytometry to assess cytotoxic T-cell (CTL) response.

During the course of treatment, the patient's lymph node size increases and later decreases as the course continues. This observation is probably due to infiltration of immune cells into the tumor site, in this case, the lymph nodes. The ELISA and flow cytometry results show an increase in antibody production and CTLs after the fourth vaccine that then persists after the completion of the series of vaccines.

### Example 2. Direct mRNA Vaccine for Equine Melanoma

A 15 year old Andalusian, presented to her veterinarian with black lesions on her neck, mane and in the perianal area. Upon review of a fine needle aspirate the pathologist diagnoses the patient with melanoma. The owner elects to pursue immunotherapy treatment due to the complicated nature of excising the perianal lesion on the patient.

Three vaccine doses are prepared containing 100 μg mRNA in 100 μL sterile nuclease free H₂O. The three doses and three needle-free injection devices (J-Tip) are shipped to the veterinarian. Three of the patient's lesions are chosen to receive the treatment course, a total of 300 μg mRNA per time point. Every two weeks three more doses are shipped to the veterinary clinic as previously done and each dose is administered to the same three lesions using the J-Tip device. The patient receives a total of six vaccine doses per lesion.

Blood samples are collected prior to initiation of the vaccine series, prior to the 5^{th} vaccine dose and two weeks after the series is completed. The blood samples are processed for peripheral blood & plasma and preserved. They are later used for evaluation of anti-tumor immune response.

Overall disease state is assessed by tumor burden reduction and anti-tumor immune response. Tumor burden is evaluated through measurements performed on the lesions before each of the six vaccine doses are administered. Anti-tumor immune response is measured using standard ELISA to assess antibody levels and flow cytometry to assess the CTL response.

As seen in other patients receiving immunotherapy treatment, the melanoma lesions will initially increase in size followed by a decrease as the vaccine series progresses. The ELISA and FACS results show an increase in antibody production and CTLs after the second vaccine that will persist after the completion of the series of vaccines.

### Example 3. Overview of Methods for emmL mRNA Creation

Methods Overview:

Restriction Enzyme Digestion of Vector and Insert

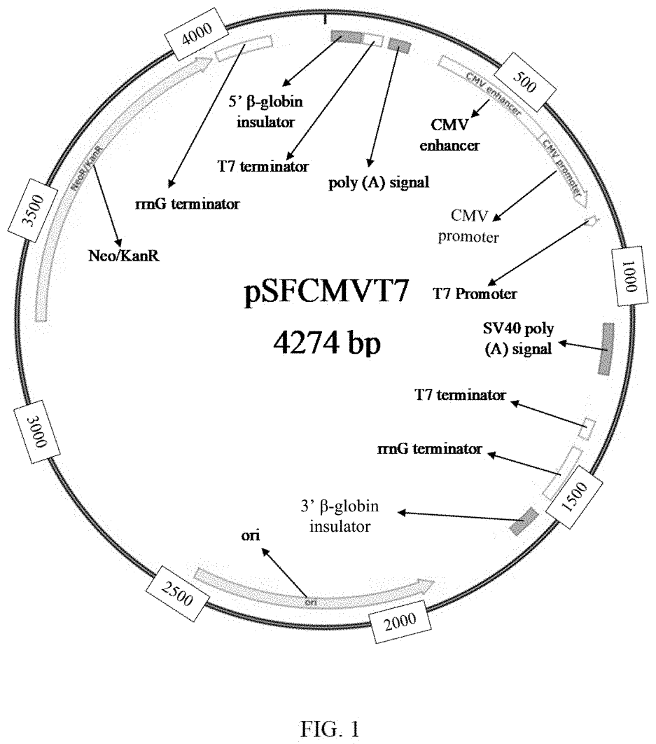

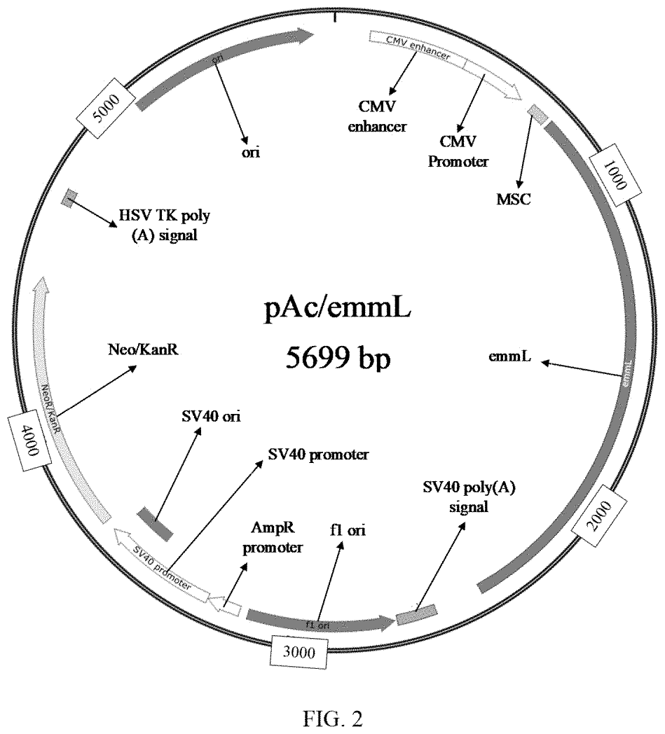

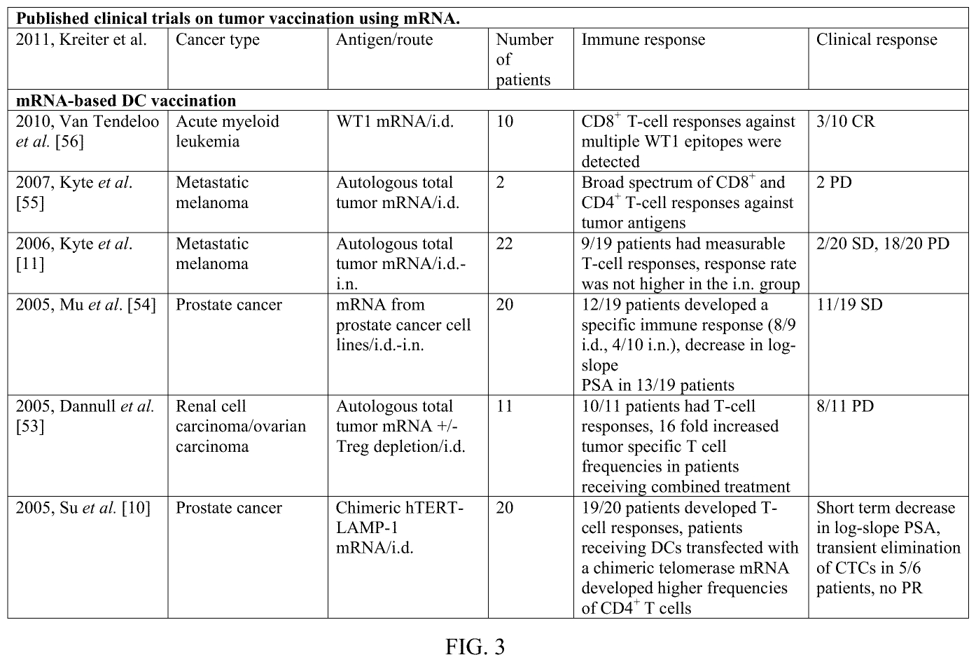

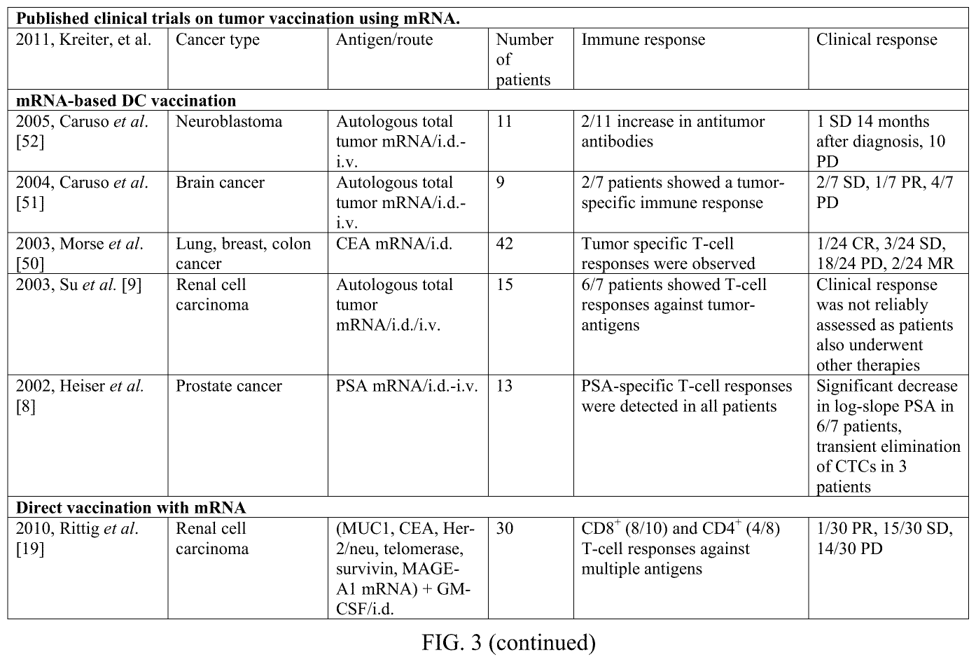

To create the appropriate recombinant plasmid for optimal mRNA production, a plasmid backbone including dual prokaryote and eukaryote promoters, untranslated 3′ and 5′ regions, and a selective marker was used. A vector of this type; for example, pSFCMVT7, has multiple features that aid in the production and stabilization of the mRNA encoding an antigenic M-like protein, such as Emm55. Both the vector pSFCMVT7 and the insert containing plasmid pAc/emmL were cut using restriction enzymes SacI and EcoRV. Refer to FIGS. 1 and 2 for plasmid maps.

DNA Fragment Separation with Gel Electrophoresis

Once the restriction digestion was performed with the appropriate enzymes, the DNA fragments were isolated through gel electrophoresis. A reference DNA ladder is run with both the digestion reactions to assess DNA band lengths, thus aiding in identification of the bands of interest. The bands containing the DNA were extracted from the gel.

Gel Extraction/DNA Isolation

The gel slices containing the DNA of interest were solubilized and the DNA extracted in order for the vector and insert to be ligated together to create the recombinant plasmid pSFCMVT7/emmL.

Vector and Insert Ligation

During the restriction digestion of the vector and insert containing plasmids, “sticky ends” were created, which were pieced together later with a ligation reaction. The “sticky ends” refer to unpaired nucleotides that are available for hydrogen bonding with the complementary nucleotides. Since the vector pSFCMVT7 and insert emmL were cut with the same restriction enzymes they contain complementary ends that were joined upon exposure to T4 DNA ligase.

Transfection into Bacteria

After the mRNA production plasmid pSFCMVT7/emmL was created, it was transformed, i.e., transfected, into competent bacteria that produce sufficient DNA that was isolated and will be used for in vitro mRNA synthesis. Invitrogen's Stb13 E. coli is an example of the type of bacteria that can be used for transfection. Transformation was induced by heat shocking the bacteria to open up small orifices in the cell membranes allowing the plasmid to enter the cell and ultimately the nucleus.

Growth and Expansion of Bacterial Culture

The bacteria transfected with the plasmid were placed onto appropriate growth medium containing a selective antibiotic. In the case of pSFCMVT7 this is a kanamycin. If the bacteria are correctly transformed with the plasmid they will produce a protein that will hinder the anti-bacterial properties of the kanamycin and allow the kanamycin-resistant bacteria to selectively grow on the medium.

Plasmid Isolation and Purification

Once an adequate number of bacteria containing pDNA had grown, the cells were lysed allowing for the plasmid to be released from inside the cells. The pDNA was isolated from the gDNA, proteins and other cellular debris through filtration and an anionic exchange column.

Preparation of Template DNA: Plasmid DNA Linearization

The isolated DNA contains the template DNA for mRNA production. In order for the transcription reaction to occur, the plasmid must be linearized. It is important that the linearization occur down-stream from the open reading frame gene of interest.

mRNA Transcription Reaction

After the template has been prepared, the message is created through an in vitro transcription reaction. This reaction simulates the transcription of mRNA in the cell, including the capping of the 5′ end and addition of a poly(A) tail for increased stabilization.

mRNA Purification

Once the message has been transcribed into mRNA, the residual DNA template is degraded so that a pure mRNA product can be used to transfect into autologous cells, allogeneic cells or intratumorally. Once inside the cells the mRNA will produce and display the M-like protein on the cell surface for immune activation.

Transfection of cancer cells with mRNA

One way the mRNA can be delivered into the cancer cells is by the method of electroporation. This method utilizes a weak electrical current that causes the cellular membrane to open up small pores that then allow the mRNA to move through the membrane and into the cytoplasm.

### Example 4. Restriction Enzyme Digestion

Table 1 shows the procedure for rapid digestion of pDNA.

TABLE 1

| STEP | | | | | |
|---|---|---|---|---|---|
| 1 | Set up two reactions as follows, each in a separate 0.5 mL | | | | |
| | tube: | | | | |
| | | pSFCMVT7 | | pAc/emmL | |
| | | (Vector) | | (Insert) | |
| | Sterile, nuclease-free water | To 50 | μL | To 50 | μL |
| | RE 10X Buffer | 5.0 | μL | 5.0 | μL |
| | Acetylated BSA (10 μg/mL) | 0.5 | μL | 0.5 | μL |
| | DNA (5 μg) | 5 | μg | 5 | μg |
| | SacI (10 μg/μL) | 3.0 | μL | 3.0 | μL |
| | EcoRV (10 μg/μL) | 3.0 | μL | 3.0 | μL |
| | Total Volume | 50.0 | μL | 50.0 | μL |

| | |
|---|---|
| 2 | Mix reaction well by pipetting up and down gently. |
| 3 | Pulse centrifuge all the tubes to get the entire contents to |
| | the bottom. |
| 4 | Incubate in dry heating block at 37° C. for 1 hour. |
| 5 | Run gel electrophoresis to separate fragments. |

### Example 5. DNA Fragment Separation with Gel Electrophoresis

Table 2 shows the procedure for DNA fragment separation.

TABLE 2

| STEP | |
|---|---|
| 1 | Dissolve 1.0% agarose in 50 mL of 1X TAE buffer by heating in |
| | the microwave. Ok it boils. Ensure all granules have solubilized. |
| 2 | Cool flask with liquid agar under running tap water. |
| 3 | Pour liquid agar into gel cast with comb making sure not to create |
| | any bubbles |
| 4 | Allow gel to solidify at room temperature. |
| 5 | While gel is cooling, Create 1 kb ladder solution by adding 10 μL |
| | Promega 1 kb ladder and 2 μL dye in a microcentrifuge tube. |
| 6 | Add 2 μL Blue juice to each reaction. |
| 7 | Load 12 μL of 1 kb ladder and 50 μL of each reaction into gel. |
| 8 | Run gel at 80 volts until samples move out of the gel wells, then |
| | increase to 100 volts until dye reaches the bottom of gel. |
| 9 | Take picture of gel using bioimaging equipment with ultraviolet |
| | light. Do not expose the gel to the UV light more than 1 minute to |
| | prevent mixing of the DNA. |
| 10 | Cut out bands of interest pSFCMVT7 vector (~4234 bp) and emmL |
| | (~1700 bp) using a sterile scalpel and store at −20° C. until needed. |

### Example 6. Gel Extraction/DNA Isolation

Table 3 shows the procedure for Extraction and DNA Isolation.

TABLE 3

| STEP | |
|---|---|
| 1 | Minimize the size of the gel slice by removing extra agarose |
| | with a scalpel. |
| 2 | Weight gel slices. |
| 3 | Add 3 volumes of gel solubilization buffer to 1 volume of gel to |
| | each. |
| 4 | Incubate at 50° C. for 10 minutes (or until the gel slice has |
| | completely dissolved). Mix by vortexing every 2-3 minutes to help |
| | dissolve gel. |
| 5 | After gel slice appears dissolved, incubate the tube for an |
| | additional 5 minutes. |
| 6 | Add 1 gel volume of isopropanol to the sample and mix. |
| 7 | Place a gel extraction column in a provided 2 mL collection tube |
| | for each sample. |
| 8 | Apply samples (maximum capacity 700 μL, meaning more than |
| | one centrifugation may need to be performed) from step 6 to the |
| | labeled columns and centrifuge at >12,000 x g for 1 minute. |
| 9 | Discard flow-through by pipetting contents out of tube, carefully |
| | avoiding leaving droplets on sides of tubes, and place column back |
| | in the same 2 mL collection tubes. |
| 10 | Add 0.5 mL of wash buffer to the columns and centrifuge |
| | at >12,000 x g for 1 minute. |
| 11 | Repeat Steps 9 and then 10. |
| 12 | Repeat Step 9 and then proceed to Step 13. |
| 13 | Remove excess buffer by centrifuging the tubes at >12,000 x g for |
| | 1 minute and repeat Step 9. |
| 14 | Remove excess ethanol by centrifuging the tubes at >12,000 x g |
| | for 3 minutes and repeat Step 9. |
| 15 | Place columns in clean 1.5 mL microcentrifuge tube. |
| 16 | Elute DNA by adding 50 μL of elution buffer to the center of each |
| | column's membrane and centrifuge for 1 minute at >12,000 x g. |
| 17 | Use spectrophotometer to measure the DNA concentration in each |
| | sample. Use these numbers to calculate the quantity of vector and |
| | insert needed for ligation. |

### Example 7. Vector and Insert Ligation

Table 4 shows the procedure for vector insert and ligation.

TABLE 4

| STEP | | | |
|---|---|---|---|
| 1 | Use the following equation to calculate the quantity of vector and insert | | |
| | needed for ligation (use molar ratio of 1:2 vector to insert): | | |
| | $\frac{ng of vector \times bp size of insert}{insert bp size of vector} \times molar ratio of \frac{insert}{vector} = ng of insert$ | | |
| 2 | Set up the following reactions in microcentrifuge tubes on ice: | | |
| | | | Negative Control |
| | | Sample | (vector only) |
| | 2 × Rapid Ligation | 5 μL | 5 μL |
| | Buffer | | |
| | Vector DNA | Calculate | Calculate |
| | Insert DNA | Calculate | n/a |
| | Nuclease-free water | To 10 μL | To 10 μL |
| | T4 DNA Ligase | 1 μL | 1 μL |
| | Total volume | 10 μL | 10 μL |

| | |
|---|---|
| 3 | Gently mix the reaction by pipetting up and down. Pulse centrifuge. |
| 4 | Incubate at 4° C. overnight (at least 12 hours). |
| 5 | Proceed to Transformation of Stbl3 E. coli protocol. |

### Example 8. Transformation of DNA into E. coli

Table 5 shows the procedure for transformation of E. coli.

TABLE 5

| STEP | |
|---|---|
| 1 | Thaw three tubes of E. coli in an ice bath immediately before using. |
| 2 | Label these tubes ligation, positive control and negative control. Negative |
| | control consists of vector only from ligation protocol. Positive control |
| | consists of the original intact plasmid. Ligation consists of the newly |
| | constructed plasmid. |
| 3 | Add 10 pg to 100 ng of the corresponding DNA to each tube. |
| 4 | Incubate tubes in an ice bath for 30 minutes. |
| 5 | Heat shock cells for 45 seconds at 42° C. |
| 6 | Return to the ice bath for 2 minutes. |
| 7 | Add 250 μL SOC medium to each tube (pre-warmed to 37° C.). |
| 8 | Cap vials tightly and shake horizontally at 37° C. and 225 rpm for 1 hour. |
| 9 | Plate 25 μL and 100 μL of each transformation, at 100% and 1:10 dilution |
| | in LB medium, onto LB with kanamycin agar plates. |
| 10 | Store remaining mix at 4° C. in case additional cells need to be plated the |
| | following day. |
| 11 | Invert and incubate the plates overnight at 37° C. |
| 12 | The following day count colonies and calculate transformation efficiency |
| | using the following equation. |
| | $\frac{\# of colonies}{X pg DNA} \times \frac{{10}^{6} pg}{μ g} \times \frac{X μ L total volume}{X μ L plated} \times dilution factor =$ |
| | transformation efficiency (# transformants/μg DNA) |
| | The efficiency should exceed 1 × 10⁸ cfu/μg plasmid. |
| 13 | Select colonies for further expansion and characterization. |

### Example 9. Growth and Expansion of Bacterial Culture

The Table 6 shows the procedure for growth and expansion of the bacterial culture.

TABLE 6

| STEP | |
|---|---|
| 1 | Select one colony from agar plate and place in culture tube with 5 |
| | mL LB broth containing kanamycin. |
| 2 | Place in 37° C. shaking incubator at 300 rpm overnight. |
| 3 | After 8-12 hours measure OD 600. When reaches 8.0 AU |
| | centrifuge bacteria and proceed according to plasmid isolation |
| | protocol. |

### Example 10. Plasmid Isolation and Purification

Table 7 shows the procedure for plasmid isolation and purification.

TABLE 7

| STEP | |
|---|---|
| 1 | Add LyseBlue reagent (one vial) to Buffer P1 and mix to yield a |
| | 1:1000 dilution. Use a sterile biological hood to add reagent. |
| 2 | Add RNase A solution (one vial) to Buffer P1 to yield a final |
| | concentration of 100 μg/mL. Use a sterile biological hood |
| | to add solution. |
| 3 | Pre-chill Buffer P1 and Buffer P3 in 4° C. refrigerator. |
| 4 | Prepare 70% ethanol by adding 3.5 mL 100% ethanol to 1.5 mL |
| | nuclease-free water. |
| 5 | Check Buffer P2 for SDS precipitation. If necessary dissolve |
| | SDS by warming to 37° C. Leave Buffer P2 bottle closed when |
| | not in use to avoid acidification from CO₂ in air. |
| 6 | Harvest the bacterial cells by centrifugation at 2773 x g for 30 |
| | minutes at 4° C. using the Beckman centrifuge. If you wish |
| | to stop the protocol and continue later, freeze cell pellets |
| | at −20° C. |
| 9 | Decant supernatant and resuspend bacterial pellet in 0.3 mL of |
| | chilled Buffer P1 (vigorously shake bottle to mix before adding |
| | to the pellet). The pellet should be completely resuspended by |
| | pipetting up and down to ensure complete mixing of lysis buffer. |
| 10 | Add 0.3 mL of Buffer P2, mix by gently inverting four to six |
| | times, and incubate at room temperature for five minutes. The |
| | solution should turn blue. |
| 11 | Add 0.3 mL of chilled Buffer P3 and mix thoroughly by inverting |
| | four to six times. The solution should turn colorless with a |
| | fluffy white precipitate. |
| 12 | Incubate on ice for 5 minutes. |
| 13 | Centrifuge at 14,000-18,000 x g for 10 minutes. Remove |
| | supernatant containing plasmid DNA. |
| 19 | Equilibrate a QIAGEN-tip 20 by applying 1 mL buffer QBT and |
| | allow the column to empty by gravity flow. |
| 20 | Apply supernatant from step 13 to the QIAGEN-tip 20 and allow |
| | column to empty by gravity flow. |
| 21 | Wash the QIAGEN-tip 20 with 2 × 2 mL buffer QC. |
| 22 | Elute DNA into a clean 1.5 mL microcentrifuge tube by adding |
| | 0.8 mL Buffer QF to the column. |
| 23 | Precipitate DNA by adding 0.7 volumes (590 μL per 800 μL |
| | of elution volume) of room-temperature isopropanol to the |
| | eluted DNA. |
| 24 | Mix and centrifuge immediately at 15,000 x g for 30 minutes. |
| | Decant supernatant. |
| 25 | Wash DNA pellet with 1 mL of 70% ethanol and centrifuge 15,000 |
| | x g for 10 minutes. Decant supernatant. |
| 26 | Air-dry pellet for 15-30 minutes and re-dissolve in suitable |
| | volume of TE buffer. Ideal final concentration is 1 mg/mL or |
| | less. |
| 26 | Measure yield of plasmid DNA following |
| 27 | Store sample at −80° C. |

### Example 11. Preparation of Template DNA

Table 8 shows the procedure for preparation of template DNA and plasmid linearization.

TABLE 8

| STEP | |
|---|---|
| 1 | Add the following to a Biopur tube (2 mL): 40 μL 10X |
| | restriction enzyme buffer, 4.0 μL BSA, 5 μg of plasmid |
| | DNA, and sterile nuclease-free water up to a final volume |
| | of 390 μL. |
| 2 | Mix by gently pipetting up and down. |
| 3 | Add 10 μL of restriction enzyme BamHI. |
| 4 | Incubate at 37° C. for 1-2 hours. |
| 5 | Terminate the digestion by adding the following into the tube |
| | in sequential order: 20 μL of 0.5M EDTA, 40 μL of 3M |
| | NH₄OAc, and 800 μL ethanol. |
| 6 | Mix and chill at −20° C. for 15 minutes. |
| 7 | Centrifuge the tube to pellet the DNA for 15 minutes at 16,000 |
| | x g and 4° C. |
| 8 | Decant the supernatant and pulse centrifuge. |
| 9 | Remove the remaining supernatant with a fine-tipped pipette. |
| 10 | Resuspend the DNA in nuclease-free water or TE buffer to yield |
| | a final concentration of 0.5 μg-1.0 μg/μL. |

### Example 12. mRNA Transcription

Table 9 shows the procedure for transcribing mRNA

TABLE 9

| STEP | | | |
|---|---|---|---|
| 1 | Thaw all frozen reagents. | | |
| 2 | Place RNA Polymerase Enzyme Mix on ice. | | |
| 3 | Vortex 10X T7 Reaction Buffer and T7 2X NTP/ARCA to | | |
| | ensure they are completely mixed. Place T7 2X NTP/ARCA | | |
| | on ice and leave 10X T7 Reaction Buffer at room temperature. | | |
| 4 | Set up the following reaction at room temperature: | | |
| | | Sample | |
| | Nuclease-free water | To 20 | μL |
| | T7 2X NTP/ARCA | 10 | μL |
| | 10X T7 Reaction Buffer | 2 | μL |
| | Linearized plasmid DNA | 0.5 | μg |
| | T7 Enzyme Mix | 2 | μL |
| | Total volume | 20 | μL |

| | |
|---|---|
| 5 | Mix gently by pipetting up and down. Pulse centrifuge. |
| 6 | Incubate at 37° C. for 1 hour. |
| 7 | Add 1 μL of TURBO DNase and mix well. |
| 8 | Incubate at 37° C. for 15 minutes. |
| 9 | To the 20 μL reaction add the following: 36 μL nuclease-free |
| | water, 20 μL 5X E-PAP Buffer, 10 μL 25 mM MnCl₂, |
| | and 10 μL ATP solution. |
| 10 | Pull 2.5 μL and set aside for gel. |
| 10 | Add 4 μL E-PAP and mix gently. |
| 11 | Incubate at 37° C. for 30-45 minutes. |
| 12 | Place reaction on ice until needed for purification reaction. |

### Example 13. mRNA Purification

Table 10 shows the procedure for purifying the mRNA

TABLE 10

| STEP | |
|---|---|
| 1 | Bring RNA sample to 100 μL with elution solution. Mix gently |
| | by pipetting up and down. |
| 2 | Add 350 μL binding solution concentrate to the sample and |
| | mix gently by pipetting up and down. |
| 3 | Add 250 μL of 100% ethanol to the sample and mix gently. |
| 4 | Insert a filter cartridge into the collection tube. |
| 5 | Add mRNA mixture to the filter cartridge. |
| 6 | Centrifuge for 1 minute at 10,000-15,000 x g. Make sure the |
| | entire mixture has passed through the filter at the end of |
| | the centrifugation. |
| 7 | Discard the flow-through and place filter back in the tube. |
| 8 | Add 500 μL wash solution and repeat step 6 and 7. |
| 9 | Repeat Step 8. |
| 10 | Pulse centrifuge the filter one more time to remove any residual |
| | wash solution. |
| 11 | Place filter in a new collection/elution tube. |
| 12 | Apply 50 μL of elution solution, close the tube cap and |
| | incubate in a heat block at 65-75° C. for 5-10 minutes. |
| 13 | Recover eluted mRNA by centrifuging for 1 minute at 10,000- |
| | 15,000 x g. |
| 14 | Measure purity and quantity using a spectrophotometer. |

### Example 14. Transfection of Cancer Cells with mRNA

Table 11 below shows the procedure for transfection of mammalian cancer cells with emmL mRNA.

TABLE 11

| STEP | |
|---|---|
| 1 | Assess the viability of the cell suspension using Trypan Blue dye |
| | exclusion. |
| 2 | For transfection collect 15 × 10⁶ cells in a 15 mL conical |
| | tube. Centrifuge cells at 638 x g and 10° C. for 10 minutes. |
| 3 | Decant supernatant and add 10 mL DPBS. Centrifuge cells at 638 |
| | x g and 10° C. for 10 minutes. |
| 4 | Repeat Step 3 two more times. |
| 5 | Resuspend cells in 300 μL transfection buffer. Mix gently using |
| | a pipette and then transfer suspension to 0.4 cm electroporation |
| | cuvette. |
| 6 | Add 20 μg mRNA to cell suspension in cuvette. Mix gently using |
| | pipette. |
| 7 | Set gene pulser to 260 v and 750 μF. Load cuvette and pulse. |
| 8 | Transfer electroporated cells in cuvette to T-75 flask with 20 mL |
| | of medium. |
| 9 | Place flask in incubator at 37° C. and 5% CO₂ overnight. |
| 10 | After 24 hours assay for expression of M-like protein on cancer |
| | cell surface. |

### Example 15. Cloning Steps for DNA pSFCMVT7/emmL

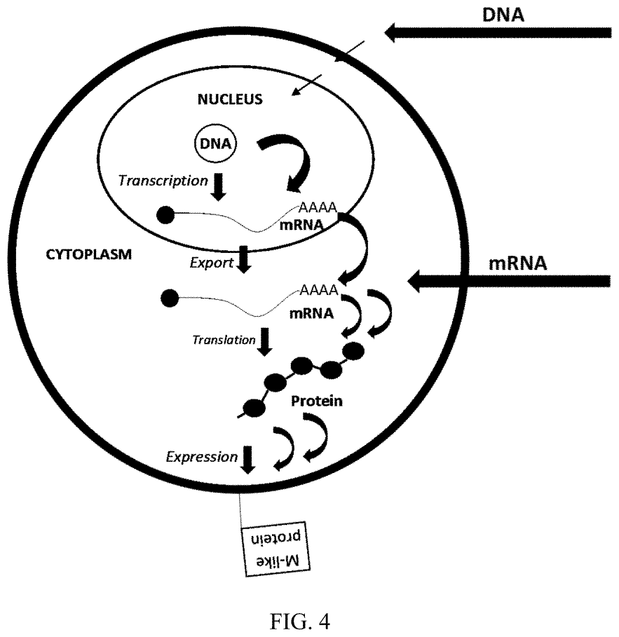

FIG. 5 shows the procedure for creating a recombinant DNA vector to produce mRNA encoding bacterial antigen.

### Example 16. Direct Binding of Antibody to M-Like Protein

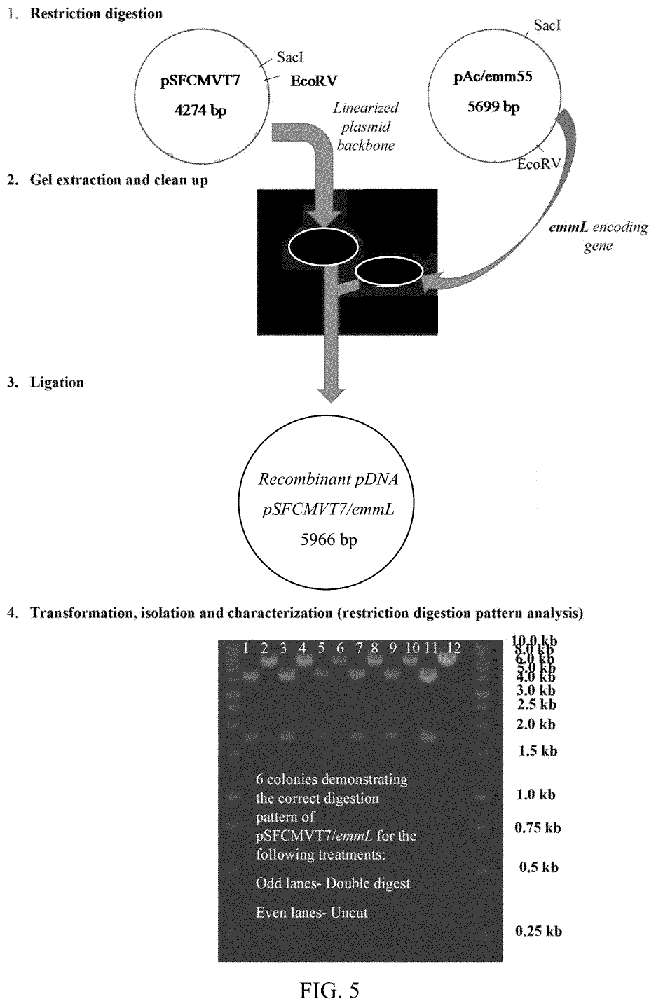

The Western blot shown in FIG. 6. demonstrates the specificity of the anti-M-like protein antibody to isolated M-like protein, specifically Emm55.

Proteins were separated by SDS-PAGE (10%) using 130 mM β-ME in the loading buffer. Samples were boiled for 3 minutes at 100° C. and spun at 13,000×g for 2 minutes at room temperature. The blots (far left) were probed with primary antibody (α-M-like Protein) for 1.5 hours at room temperature in 5% milk. The primary antibody dilution was 1:500. The secondary antibody (goat α-mouse conjugated HRP) was at a dilution of 1:5000. The null blot (second from left) shows the non-specific binding of secondary antibody.

Chemiluminescence was used to visualize the protein on the nitrocellulose blots (exposure: 10 minutes).

### Example 17. Fluorescence Microscope Images and Chart Demonstrating the Increased

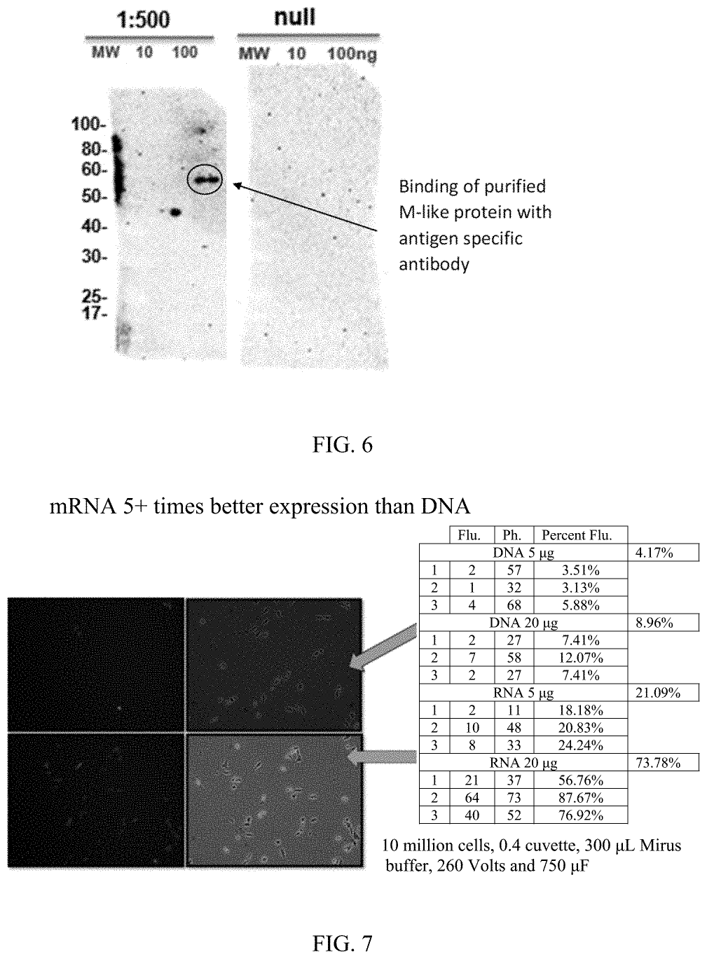

expression seen with mRNA as compared with DNA. Results were compared from an experiment in which RNA and DNA were transfected into mammalian cells and assayed for protein expression. The results show that RNA at equivalent transfection quantity produced five times the amount of expression. (See FIG. 7).

### Example 18. Synthesized emmL mRNA, Untailed and Tailed

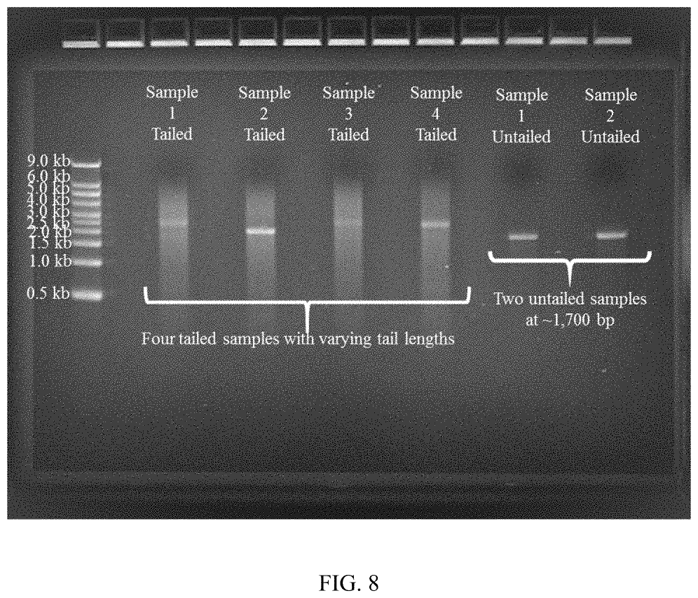

The procedure used to perform the denaturing agarose gel shown in FIG. 8 demonstrates the visualization of the synthesized emmL mRNA, specifically emm55 mRNA using the Lonza FlashGel system.

20 ng samples and 100 ng ladder were prepared by diluting the total quantity into a 2.5 μL volume using DEPC treated water. An equivalent volume of formaldehyde sample buffer was added to each sample. The samples were mixed and then incubated at 65° C. for 15 minutes followed by a 1 minute incubation on ice. Samples were loaded into a 1.2% RNA gel cassette and then run at 225 volts for 8 minutes. The gel was incubated at room temperature for 10 minutes and then visualized using the FlashGel camera. mRNA sizes are determined by the RNA Millennium Marker.

### Example 19

Chart 4 displays the results from an experiment in which RNA (emmL mRNA) and DNA (pSFCMVT17/emmL) were transfected into mammalian cells, stained with α-M-like protein, and assayed using flow cytometric analysis. The results show that the RNA-transfected cells produce an equivalent signal to the DNA transfected cells, i.e. 9%.

CHART 4

| | | | |
|---|---|---|---|
| | | | Minus Stained |
| Tube Treatment | % Parent | Minus Unstained | Untransfected |

Unstained

| | |
|---|---|
| Untransfected | 1 |
| emmL mRNA | 0.4 |
| pSFCMVT17/emmL | 1.9 |

Stained

| | | | |
|---|---|---|---|
| Untransfected | 5.6 | 4.6 | −1 |
| emmL mRNA | 14.8 | 14.4 | 9 |
| pSFCMVT17/emmL | 16.6 | 14.7 | 9 |

### Example 20

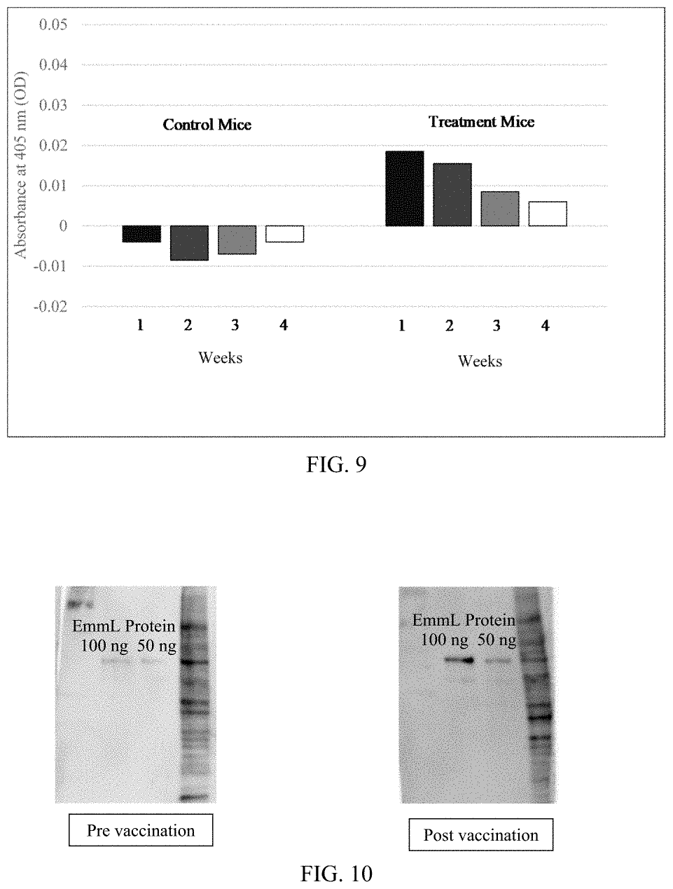

Blood samples from mice vaccinated with either emmL mRNA (treatment) or sterile water (control) were tested for the presence of antibodies that react to emmL protein. As shown in FIG. 9, the blood sample from the control mice (C2) did not contain α-M-like Protein antibodies, whereas the treatment mice (T2) sample showed a slight elevation.

### Example 21

Chart 5 shows the results from an experiment in which mice were transplanted with melanoma tumor cells and subsequently injected with either emmL mRNA (treatment) or sterile water (control). The injection regimen began 10 days after tumor implantation. The regimen consisted of three injections, of either treatment or control, administered every seven days. All five mice in the experiment lived past injection #2. At this time, two out of three treatment mice had smaller tumors than the control mice. Three of the five mice survived past injection #3, at which point the two remaining treatment mice tumors were still smaller than the remaining control mouse tumor.

CHART 5

Tumor Measurements (mm²)

| | |
|---|---|
| Post Injection #2 | Post Injection #3 |

Treatment

| | | |
|---|---|---|
| 1 | 44 | 163 |
| 2 | 102 | 100 |
| 3 | 235 | n/a |

Control

| | | |
|---|---|---|
| 1 | 115 | 193 |
| 2 | 188 | n/a |

### Example 22

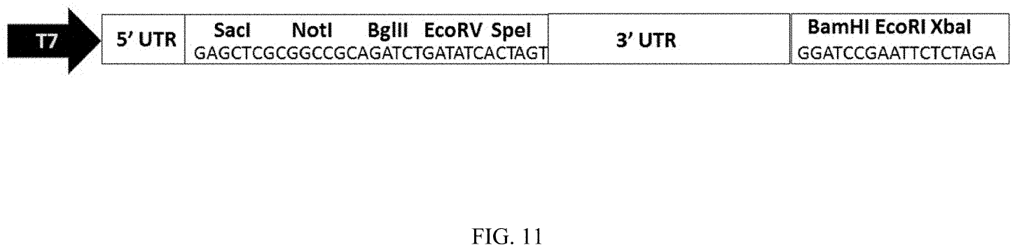

FIG. 10 shows results from an experiment in which blood samples from mice, pre and post vaccination with emmL mRNA, were tested for the presence of antibodies that react to emmL protein. The Western blot images indicate that the blood samples taken post-vaccination have increased binding of antibodies from the pre-vaccination sample.

### Example 23

A PCR with high fidelity Taq DNA polymerase is used to amplify the emmL coding region from the pAc/emm55 plasmid. In one embodiment, this amplimer is designed with proximal elements that include restriction endonuclease sites compatible with the pSF-CMV_T7 vector to minimize potentially inhibitory elements within the 5′ and 3′ UTRs of pSFCMVT7/emmL.

The proximal design elements include restriction endonuclease recognition sites for NcoI and XhoI, an optimal translation initiation sequence (SEQ ID NO: 4 or 5) and two additional stop codons (SEQ ID NO: 6) downstream of the emmL coding region, with respect to the activity of T7 RNA polymerase. The resultant amplimer can be inserted into a PCR cloning vector such as pCR®II-TOPO. The resultant plasmid can be used to transform E. coli, select for positive transformation on an agar plates containing a selective antibiotic that matches the PCR cloning vector, such as agar plates containing Luria Bertani broth supplemented with 50-100 μg/mL kanamycin or carbenicillin in the case of pCR®II-TOPO.

Bacterial cultures containing the resultant plasmid can expanded by growth in a liquid antibiotic selective media, such as Luria Bertani broth supplemented with 50-100 μg/mL kanamycin or carbenicillin in the case of pCR II-TOPO. Plasmid DNA can be prepared from these bacterial cultures using methods known to a person practiced in the art. The restriction endonucleases NcoI and XhoI can then be used to excise the emmL coding region flanked by proximal design elements from pCR II-TOPO plasmid DNA. The emmL coding region flanked by proximal design elements DNA fragment can then be inserted into the pSF-CMV_T7 vector, which has been digested with NcoI and XhoI. After ligation, bacterial transformation, selection for positive transformation on an agar plates containing 50-100 μg/mL kanamycin, expansion in Luria Bertani broth supplemented with 50-100 μg/mL kanamycin and plasmid DNA preparation using methods known to a person practiced in the art, the resultant plasmid, pSF/emmL can be used as a template for in vitro mRNA synthesis following linearization with the restriction endonuclease XhoI. The resultant mRNA and predicted amino acid sequences are shown as SEQ ID NOs: 13 and 14, respectively.

### Example 24

High fidelity Taq DNA polymerase is used to amplify the emmL coding region from the pAc/emm55 plasmid with proximal design elements that include restriction endonuclease sites compatible with the polylinker region of pT7XLUTR. In one embodiment, this can also include an optimal translation initiation sequence (SEQ ID NO: 4 or 5) and two additional stop codons (SEQ ID NO: 6). The full sequence of this embodiment is shown in SEQ ID 15. The resulting amplimer can be inserted into a PCR cloning vector such as pCR®II-TOPO. Following bacterial transformation, selection, expansion and plasmid DNA preparation described in example 23, the resultant plasmid can be digested with restriction endonucleases SacI and SpeI to excise the emmL coding region with proximal design elements from pCR®II-TOPO. The emmL coding region with proximal design elements can then be ligated into the pT7XLUTR plasmid vector that has been digested with SacI and SpeI to generate pT7XLUTR/emmL. The resultant pT7XLUTR/emmL plasmid can be used to transform E. coli.

Following selection, expansion and plasmid DNA preparation described in example 23, the resultant pT7XLUTR/emmL plasmid can be used as a template for in vitro mRNA synthesis following linearization with the restriction endonuclease BamHI. The resultant mRNA and predicted amino acid sequences are shown as SEQ ID NOs: 15 and 14, respectively.

### Example 25

High fidelity Taq DNA polymerase is used to amplify the emmL coding region from the pAc/emm55 plasmid with minimal 5′ and 3′ UTRs. The T7 RNA polymerase promoter sequence (SEQ. ID NO 8) is added as a proximal design element along with an optimal translation initiation sequence (SEQ ID NO: 4 or 5), two additional stop codons (SEQ ID NO: 6) and an XhoI restriction endonuclease site.

The resultant PCR product can be inserted into a PCR cloning vector such as pCR®II-TOPO. Following bacterial transformation, selection, expansion and plasmid DNA preparation described in example 23, the resultant plasmid, pT7/emmL can be used as a template for in vitro mRNA synthesis following linearization with the restriction endonuclease XhoI. The resultant mRNA and predicted amino acid sequences are shown as SEQ ID NOs: 16 and 14, respectively.

In examples 23-25, mRNA encoding emmL can be produced using an in vitro transcription reaction. Several modifications to the resultant mRNA can be made in this reaction to improve mRNA and EmmL protein stability and translation efficiency, and to reduce mRNA immunogenicity. For example, a modified nucleic acid can be attached to the 5′ end of emmL mRNA such as, but not limited to, anti-reverse Cap Analog [ARCA, P1-(5′-(3′-O-methyl)-7-methyl-guanosyl) P3-(5′-(guanosyl))triphosphate)], N1-methyl-guanosine, 2′ fluoro-guanosine, 7-deaza-guanosine, inosine, 8-oxo-guanosine, 2-amino-guanosine, LNA-guanosine and 2-azido-guanosine.

In another example, a polyadenylated [poly(A)] tail approximately 50-200 adenosine monophosphates in length can be attached to the 3′ end of emmL mRNA or both 5′ modified nucleotide cap and poly(A) tail can be added to emmL mRNA.

emmL mRNA can be synthesized with ribonucleotide analogs. Chemical modifications can be made at this stage to further improve translation efficiency and stability. Examples include; 5-methyl-cytidine-5′-triphosphate, pseudouridine-5′-triphosphate, 2-thiouridine-5′triphosphate, and N1-methylpseudouridine-5′-triphosphate.

SEQ ID NO: 4. Degenerate DNA sequence of an optimal translation initiation sequence. R is A or G, Y is C or T, M is A or C and V is A, C or G.

RYMRMVATGGC

SEQ ID NO: 5. DNA sequence of an optimal translation initiation sequence.

ATAGCCATGGC

SEQ ID NO: 6. DNA sequence containing 2 stop codons.

TAATGA

SEQ ID NO: 7. DNA sequence of the restriction endonuclease recognition sites for SacI, NotI, BglII, EcoRV and SpeI.

GAGCTCGCGGCCGCAGATCTGATATCACTAGT

SEQ ID NO: 8. DNA sequence of the T7 RNA polymerase promoter.

TAATACGACTCACTATAG

SEQ ID NO: 9. 50 ribonucleotides of the Xenopus laevis beta globin gene 5′ untranslated region.

aagcuucuuguucuuuuugcagaagcucagaauaaacgcucaacuuuggc

SEQ ID NO: 10. 74 ribonucleotides of the Xenopus laevis beta globin gene 3′ untranslated region.

cuuuuugaugccauugccgacgcccuuggcaaggguuaccacuaaaccag
ccucaagaacacccgaauggaguc

SEQ ID NO: 11. DNA sequence of the restriction endonuclease recognition sites for BamHI, EcoRI and XbaI.

GGATCCGAATTCTCTAGA

SEQ ID NO: 12. DNA sequence of the synthetic double stranded DNA molecule used to prepare pT7XLUTR. T7 RNA polymerase, restriction endonuclease recognition sites for SacI, NotI, BglII, EcoRV, SpeI, as well as BamHI, EcoRI and XbaI are underlined.

TAATACGACTCACTATAGAAGCTTCTTGTTCTTTTTGCAGAAGCTCAGAA
TAAACGCTCAACTTTGGCGAGCTCGCGGCCGCAGATCTGATATCACTAGT
CTTTTTGATGCCATTGCCGACGCCCTTGGCAAGGGTTACCACTAAACCAG
CCTCAAGAACACCCGAATGGAGTCGGATCCGAATTCTCTAGA

SEQ ID NO: 13 (RNA sequence of example 23). Synthetic mRNA sequence following in vitro mRNA synthesis of emmL from a plasmid template, written from 5′ to 3′ with respect to the mRNA molecule. An anti-reverse cap analog (ARCA) is added to the 5′ end of the transcript and approximately 50-200 adenosine monophosphates (a⁵⁰⁻²⁰⁰) are added to the 3′ end of the transcript during in vitro transcription. 5′ lowercase letters are the 5′ untranslated region (UTR) from the pSF-T7_CMV plasmid vector. The guanine (g) immediately after the ARCA cap is the +1 ribonucleotide produced by T7 RNA polymerase. Bold indicates ribonucleotides added to make the 5′ NcoI restriction endonuclease site, which is underlined, and an optimal translation initiation sequence. Uppercase letters indicate the coding sequence of emmL. At the 3′ end of the mRNA sequence, bold indicates two additional stop codons. The 3′ XhoI restriction endonuclease site is underlined.

gggagaagaucuuugucgauccuaccauccacucgacacacccgccagcg
gccgcugccaagcuuccgagcucaagcuucgaauucugcagucgacggua
ccgcgggcccgggauccauaaggagcauaaaaauagccAUGGCUAAAAAU
ACCACGAAUAGACACUAUUCGCUUAGAAAAUUAAAAACAGGAACGGCUUC
AGUAGCAGUAGCUUUGACUGUUUUAGGGACAGGACUGGUAGCAGGGCAGA
CAGUAAAAGCAAGCCAAACAGAACCAUCUCAGACCAAUAACAGAUUAUAU
CAAGAAAGACAACGUUUACAGGAUUUAAAAAGUAAGUUUCAAGACCUGAA
AAAUCGUUCAGAGGGAUACAUUCAGCAAUACUACGACGAAGAAAAGAACA
GUGGAAGUAACUCUAACUGGUACGCAACCUACUUAAAAGAAUUAAAUGAC
GAAUUUGAACAAGCUUAUAAUGAACUUAGUGGUGAUGGUGUAAAAAAAUU
AGCUGCAAGUUUGAUGGAAGAAAGAGUCGCUUUAAGAGACGAAAUCGAUC
AGAUUAAGAAAAUAUCAGAAGAAUUAAAAAAUAAGCUGAGAGCAAAAGAA
GAAGAAUUAAAAAAUAAAAAAGAGGAACGUGAGCUUGAGCAUGCUGCCUA
UGCAGCAGAUGCAAAGAAACAUGAAGAAUAUGUCAAAUCCAUGUCUCUCG
UACUAAUGGAUAAAGAAGAGGAGCGUCAUAAACUAGAGCAAUCAUUAGAC
ACGGCUAAAGCUGAGCUUGUUAAAAAAGAGCAAGAGUUACAGUUAGUCAA
AGGCAAUCUAGAUCAAAAAGAAAAAGAACUAGAAAAUGAAGAGCUAGCGA
AAGAAAGUGCUAUUAGUGAUUUGACUGAGCAGAUUACUGCUAAGAAGGCU
GAAGUAGAAAAAUUAACUCAAGAUUUAGCUGCUAAGUCUGCUGAAAUUCA
GGAAAAAGAAGCUGAAAAAGAUCGCCAACAGCAUAUGUACGAAGCGUUUA
UGAGCCAGUACAAAGAAAAAGUUGAGAAACAAGAGCAAGAGCUUGCUAAG
CUAAAACAACUUGAAACCAUCAACAACAAUCUAUUAGGUAAUGCUAAGGA
UAUGAUAGCUAAGUUGUCUGCUGAAAAUGAACAAUUAGCAAGCGACAAAG
CAAAACUUGAAGAACAAAACAAGAUUUCAGAAGCGAGCCGUAAAGGUCUU
CGUCGUGACUUGGACGCAUCACGUGAAGCUAAGAAACAAGUUGAAAAAGA
UUUAGCAAACUUGACUGCUGAACUUGAUAAGGUUAAAGAAGAUAAACAAA
UUUCAGACGCAAGCCGUAAAGGUCUUCGUCGUGACUUGGACGCAUCACGU
GAAGCUAAGAAACAAGUUGAAAAAGCUUUAGAAGAAGCAAACAGCAAAUU
AGCGGCUCUUGAAAAACUUAACAAAGAGCUUGAAGAAAGCAAGAAAUUAA
CAGAAAAAGAAAAAGCUGAGCUACAAGCGAAACUUGAAGCAGAAGCAAAA
GCACUCAAAGAACAAUUAGCGAAACAAGCUGAAGAACUUGCAAAACUAAG
AGCUGGAAAAGCAUCAGACUCACAAACCCCUGAUGCAAAACCAGGAAACA
AAGUUGUUCCAGGUACAGGUCAAGCACCACAAGCAGGCACAAAACCUAAC
CAAAACAAAGCACCAAUGAAGGAAACUAAGAGACAGUUACCAUCAACAGG
UGAAGCAGCUAAUCCAUUCUUUACAGCGGCAGCCCUUACUGUUAUGGCAA
CAGCUGGAGUAGCAGCAGUUGUAAAACGCAAAGAAGAAAACGAAGCUGAA
UUCUGCAGAUAUCCAUCACACUGGCGGCCGCGACUCUAGuaaugacucga
g (a⁵⁰⁻²⁰⁰)

SEQ ID NO: 14 (Predicted protein sequence of examples 23-25). One letter predicted amino acid sequence of emmL transcripts produced in examples 2-4. * indicates stop codon.

MAKNTTNRHYSLRKLKTGTASVAVALTVLGTGLVAGQTVKASQTEPSQTN
NRLYQERQRLQDLKSKFQDLKNRSEGYIQQYYDEEKNSGSNSNWYATYLK
ELNDEFEQAYNELSGDGVKKLAASLMEERVALRDEIDQIKKISEELKNKL
RAKEEELKNKKEERELEHAAYAADAKKHEEYVKSMSLVLMDKEEERHKLE
QSLDTAKAELVKKEQELQLVKGNLDQKEKELENEELAKESAISDLTEQIT
AKKAEVEKLTQDLAAKSAEIQEKEAEKDRQQHMYEAFMSQYKEKVEKQEQ
ELAKLKQLETINNNLLGNAKDMIAKLSAENEQLASDKAKLEEQNKISEAS
RKGLRRDLDASREAKKQVEKDLANLTAELDKVKEDKQISDASRKGLRRDL
DASREAKKQVEKALEEANSKLAALEKLNKELEESKKLTEKEKAELQAKLE
AEAKALKEQLAKQAEELAKLRAGKASDSQTPDAKPGNKVVPGTGQAPQAG
TKPNQNKAPMKETKRQLPSTGEAANPFFTAAALTVMATAGVAAVVKRKEE
NEAEFCRYPSHWRPRL*

SEQ ID NO: 15 (RNA sequence of example 24). Synthetic mRNA sequence following in vitro mRNA synthesis of emmL from the pT7XLURT plasmid template linearized with restriction endonuclease BamHI, written from 5′ to 3′ with respect to the mRNA molecule. An anti-reverse cap analog (ARCA) is added to the 5′ end of the transcript and approximately 50-200 adenosine monophosphates (a⁵⁰⁻²⁰⁰) are added to the 3′ end of the transcript during in vitro transcription. The guanine (g) immediately after the ARCA cap is the +1 ribonucleotide produced by T7 RNA polymerase. 5′ lowercase letters are from the 5′ untranslated region of the Xenopus laevis beta globin gene. Bold indicates ribonucleotides added to make an optimal translation initiation sequence. A SacI restriction endonuclease site 5′ of the emmL coding region is underlined. Uppercase letters indicate the coding sequence of emmL. Lowercase letters 3′ of the coding region are the 3′ untranslated region of the Xenopus laevis beta globin gene. At the 3′ end of the mRNA sequence, bold indicates two additional stop codons. SpeI and BamHI restriction endonuclease sites are underlined.

gaagcuucuuguucuuuuugcagaagcucagaauaaacgcucaacuuugg
cgagcucauagccAUGGCUAAAAAUACCACGAAUAGACACUAUUCGCUUA
GAAAAUUAAAAACAGGAACGGCUUCAGUAGCAGUAGCUUUGACUGUUUUA
GGGACAGGACUGGUAGCAGGGCAGACAGUAAAAGCAAGCCAAACAGAACC
AUCUCAGACCAAUAACAGAUUAUAUCAAGAAAGACAACGUUUACAGGAUU
UAAAAAGUAAGUUUCAAGACCUGAAAAAUCGUUCAGAGGGAUACAUUCAG
CAAUACUACGACGAAGAAAAGAACAGUGGAAGUAACUCUAACUGGUACGC
AACCUACUUAAAAGAAUUAAAUGACGAAUUUGAACAAGCUUAUAAUGAAC
UUAGUGGUGAUGGUGUAAAAAAAUUAGCUGCAAGUUUGAUGGAAGAAAGA
GUCGCUUUAAGAGACGAAAUCGAUCAGAUUAAGAAAAUAUCAGAAGAAUU
AAAAAAUAAGCUGAGAGCAAAAGAAGAAGAAUUAAAAAAUAAAAAAGAGG
AACGUGAGCUUGAGCAUGCUGCCUAUGCAGCAGAUGCAAAGAAACAUGAA
GAAUAUGUCAAAUCCAUGUCUCUCGUACUAAUGGAUAAAGAAGAGGAGCG
UCAUAAACUAGAGCAAUCAUUAGACACGGCUAAAGCUGAGCUUGUUAAAA
AAGAGCAAGAGUUACAGUUAGUCAAAGGCAAUCUAGAUCAAAAAGAAAAA
GAACUAGAAAAUGAAGAGCUAGCGAAAGAAAGUGCUAUUAGUGAUUUGAC
UGAGCAGAUUACUGCUAAGAAGGCUGAAGUAGAAAAAUUAACUCAAGAUU
UAGCUGCUAAGUCUGCUGAAAUUCAGGAAAAAGAAGCUGAAAAAGAUCGC
CAACAGCAUAUGUACGAAGCGUUUAUGAGCCAGUACAAAGAAAAAGUUGA
GAAACAAGAGCAAGAGCUUGCUAAGCUAAAACAACUUGAAACCAUCAACA
ACAAUCUAUUAGGUAAUGCUAAGGAUAUGAUAGCUAAGUUGUCUGCUGAA
AAUGAACAAUUAGCAAGCGACAAAGCAAAACUUGAAGAACAAAACAAGAU
UUCAGAAGCGAGCCGUAAAGGUCUUCGUCGUGACUUGGACGCAUCACGUG
AAGCUAAGAAACAAGUUGAAAAAGAUUUAGCAAACUUGACUGCUGAACUU
GAUAAGGUUAAAGAAGAUAAACAAAUUUCAGACGCAAGCCGUAAAGGUCU
UCGUCGUGACUUGGACGCAUCACGUGAAGCUAAGAAACAAGUUGAAAAAG
CUUUAGAAGAAGCAAACAGCAAAUUAGCGGCUCUUGAAAAACUUAACAAA
GAGCUUGAAGAAAGCAAGAAAUUAACAGAAAAAGAAAAAGCUGAGCUACA
AGCGAAACUUGAAGCAGAAGCAAAAGCACUCAAAGAACAAUUAGCGAAAC
AAGCUGAAGAACUUGCAAAACUAAGAGCUGGAAAAGCAUCAGACUCACAA
ACCCCUGAUGCAAAACCAGGAAACAAAGUUGUUCCAGGUACAGGUCAAGC
ACCACAAGCAGGCACAAAACCUAACCAAAACAAAGCACCAAUGAAGGAAA
CUAAGAGACAGUUACCAUCAACAGGUGAAGCAGCUAAUCCAUUCUUUACA
GCGGCAGCCCUUACUGUUAUGGCAACAGCUGGAGUAGCAGCAGUUGUAAA
ACGCAAAGAAGAAAACGAAGCUGAAUUCUGCAGAUAUCCAUCACACUGGC
GGCCGCGACUCUAGuaaugaacuagucuuuuugaugccauugccgacgcc
cuuggcaaggguuaccacuaaaccagccucaagaacacccgaauggaguc
ggaucc (a⁵⁰⁻²⁰⁰)

SEQ ID NO: 16 (RNA sequence of example 24). Synthetic mRNA sequence following in vitro mRNA synthesis of emmL from a plasmid template, written from 5′ to 3′ with respect to the mRNA molecule. An anti-reverse cap analog (ARCA) is added to the 5′ end of the transcript and approximately 50-200 adenosine monophosphates (a⁵⁰⁻²⁰⁰) are added to the 3′ end of the transcript during in vitro transcription. The guanine (g) immediately after the ARCA cap is the +1 ribonucleotide produced by T7 RNA polymerase. Bold indicates ribonucleotides added to make an optimal translation initiation sequence. Uppercase letters indicate the coding sequence of emmL. At the 3′ end of the mRNA sequence, bold indicates two additional stop codons. The 3′ XhoI site is underlined.

gauagccAUGGCUAAAAAUACCACGAAUAGACACUAUUCGCUUAGAAAAU
UAAAAACAGGAACGGCUUCAGUAGCAGUAGCUUUGACUGUUUUAGGGACA
GGACUGGUAGCAGGGCAGACAGUAAAAGCAAGCCAAACAGAACCAUCUCA
GACCAAUAACAGAUUAUAUCAAGAAAGACAACGUUUACAGGAUUUAAAAA
GUAAGUUUCAAGACCUGAAAAAUCGUUCAGAGGGAUACAUUCAGCAAUAC
UACGACGAAGAAAAGAACAGUGGAAGUAACUCUAACUGGUACGCAACCUA
CUUAAAAGAAUUAAAUGACGAAUUUGAACAAGCUUAUAAUGAACUUAGUG
GUGAUGGUGUAAAAAAAUUAGCUGCAAGUUUGAUGGAAGAAAGAGUCGCU
UUAAGAGACGAAAUCGAUCAGAUUAAGAAAAUAUCAGAAGAAUUAAAAAA
UAAGCUGAGAGCAAAAGAAGAAGAAUUAAAAAAUAAAAAAGAGGAACGUG
AGCUUGAGCAUGCUGCCUAUGCAGCAGAUGCAAAGAAACAUGAAGAAUAU
GUCAAAUCCAUGUCUCUCGUACUAAUGGAUAAAGAAGAGGAGCGUCAUAA
ACUAGAGCAAUCAUUAGACACGGCUAAAGCUGAGCUUGUUAAAAAAGAGC
AAGAGUUACAGUUAGUCAAAGGCAAUCUAGAUCAAAAAGAAAAAGAACUA
GAAAAUGAAGAGCUAGCGAAAGAAAGUGCUAUUAGUGAUUUGACUGAGCA
GAUUACUGCUAAGAAGGCUGAAGUAGAAAAAUUAACUCAAGAUUUAGCUG
CUAAGUCUGCUGAAAUUCAGGAAAAAGAAGCUGAAAAAGAUCGCCAACAG
CAUAUGUACGAAGCGUUUAUGAGCCAGUACAAAGAAAAAGUUGAGAAACA
AGAGCAAGAGCUUGCUAAGCUAAAACAACUUGAAACCAUCAACAACAAUC
UAUUAGGUAAUGCUAAGGAUAUGAUAGCUAAGUUGUCUGCUGAAAAUGAA
CAAUUAGCAAGCGACAAAGCAAAACUUGAAGAACAAAACAAGAUUUCAGA
AGCGAGCCGUAAAGGUCUUCGUCGUGACUUGGACGCAUCACGUGAAGCUA
AGAAACAAGUUGAAAAAGAUUUAGCAAACUUGACUGCUGAACUUGAUAAG
GUUAAAGAAGAUAAACAAAUUUCAGACGCAAGCCGUAAAGGUCUUCGUCG
UGACUUGGACGCAUCACGUGAAGCUAAGAAACAAGUUGAAAAAGCUUUAG
AAGAAGCAAACAGCAAAUUAGCGGCUCUUGAAAAACUUAACAAAGAGCUU
GAAGAAAGCAAGAAAUUAACAGAAAAAGAAAAAGCUGAGCUACAAGCGAA
ACUUGAAGCAGAAGCAAAAGCACUCAAAGAACAAUUAGCGAAACAAGCUG
AAGAACUUGCAAAACUAAGAGCUGGAAAAGCAUCAGACUCACAAACCCCU
GAUGCAAAACCAGGAAACAAAGUUGUUCCAGGUACAGGUCAAGCACCACA
AGCAGGCACAAAACCUAACCAAAACAAAGCACCAAUGAAGGAAACUAAGA
GACAGUUACCAUCAACAGGUGAAGCAGCUAAUCCAUUCUUUACAGCGGCA
GCCCUUACUGUUAUGGCAACAGCUGGAGUAGCAGCAGUUGUAAAACGCAA
AGAAGAAAACGAAGCUGAAUUCUGCAGAUAUCCAUCACACUGGCGGCCGC
GACUCUAGuaaugacucgag (a⁵⁰⁻²⁰⁰)

### Example 26

Codon optimization algorithms were also used to synthesize emm55 mRNAs. Emm55 expression was measured and compared to determine relative increase protein synthesis and stability in mammalian cells. Each mRNA was synthesized in vitro using wild type uridine (WT) and N1-methylpseudouridine (N1) was shown to improve protein expression.

Three different algorithms were used to optimize the nucleotide sequence of emm55 from expression in Streptococcus pyogenes to expression in humans. Codon optimization was carried out using the JCat algorithm (Grote 2005) and two proprietary algorithms run by the Karolinska Institute (FreqDist and MostFreq). mRNAs designed by these three algorithms were synthesized by TriLink® Biotechnologies in vitro using both wild type (WT) uridine and chemically modified N1-methyl pseudouridine (N1), which can increase mRNA stability and protein expression levels in vitro (Svitkin 2017) and in vivo (Pardi 2015). The final nucleotide sequence for each mRNA is shown as SEQ ID NO: 17, SEQ ID NO: 18 and SEQ ID NO: 19. As a part of the design, BbsI and BspQI restriction endonuclease sites were removed by base substitution from emm55JCat and emm55MostFreq to facilitate cloning into the TriLink® plasmid vector used to synthesize mRNA in vitro.

mRNAs were received with each tube of mRNA supplied by TriLink® Biotechnologies and added as a stock item so that each tube could be tracked independently. mRNAs were stored at −80° C. Quality control testing was carried out to verify purity and intactness using RD 3-75.1, except that mRNAs were aliquoted by TriLink® Biotechnologies rather than as specified in the SOP (standard operating procedure).

mRNAs were transiently transfected into the attachment enhanced HEK subline HEK293T-AE and B16-F10 cells using RD 3-79.1 (2.5 μg mRNA/well). Cells were transfected with pAc/emm55 using RD 3-34.2 (4 μg pDNA/well). Two to 48 hours post transfection, cells were lysed in RIPA buffer using RD 3-83.1 and the protein concentrations of the lysates was determined by BCA assay using RD 3-14.2.

EGFP mRNA and pDNA were used as transfection positive controls and Emm55 expression negative controls. Initially, lysates were resolved using denaturing SDS-PAGE, western blotted, stained with chicken anti-Emm55 and detected using chemiluminescence using RD 3-74.1. The band corresponding in size to Emm55 consistently overwhelmed the peroxidase detection reaction, resulting in bands that could not be quantified. No suitable dilution of chicken anti-Emm55 or goat anti-chicken-horseradish peroxidase could be identified to increase the high end of the detection range. For this reason, Emm55 protein detection was carried out using the ProteinSimple Wes™ capillary electrophoresis system using chicken anti-Emm55. This system resolves protein and protein/antibody complexes in a reducing SDS-PAGE capillary tube to provide antibody binding quantification like western blot data. These assays were carried out using manufacturer-supplied protocol.

The Wes™ assay measured the level of Emm55 in cell lysate normalized to the level of ERK1, a serine/threonine kinase that is regulated by phosphorylation rather than protein level, and thus makes a suitable protein for data normalization within a cell or tissue type. This approach has an advantage over internal total protein normalization, as it can be multiplexed with Emm55 detection, providing a sample loading control and cutting in half the number of sample assays that need to be performed. To establish the linearity of this assay, a standard curve was constructed with 10 ng to 30 pg rEmm55 in 1.5 μg untransfected HEK293T-AE lysate. Antibody dilutions for the Wes™ assay were; chicken anti-Emm55 at 1:100 and goat anti-chicken-HRP at 1:100.

The Wes™ assay assembly instructions refer to input protein concentration rather than mass. Experiments using the Wes™ were carried out using cell lysates at 0.1, 0.25 and 0.5 μg/μL. While 0.5 μg/μL gave results within the Wes™ dynamic range with B16-F10 cell lysates, 0.25 and 0.5 HEK293T lysates generated expression levels that exceeded the detection capacity at peak expression time points. A concentration of 0.1 μg/μL lysate was found to be optimal throughout the time course, although lower concentrations could be used to provide additional dynamic range when high level expression is anticipated. However, this lower input concentration may miss low but biologically meaningful levels of expression, especially at early and late time points.

Data normalization was carried out by dividing the Wes™ numerical output value for Emm55 by the output value for ERK1. In experiments with data replication, normalization was carried out on individual samples then mean±standard deviation values (n=3) were calculated. At one timepoint (0.1 μg/μL emm55JCat-WT 12 hrs post transfection), an anomalously low ERK1 level resulted in a single dropped normalized datapoint based on Grubbs' Extreme Studentized Deviate test. In this case the mean was calculated from the remaining two replica data points. A one-way ANOVA with both Bonferroni post-hoc test was performed using GraphPad Prism software to determine significant differences between means. Significant findings were confirmed using the Tukey post-hoc test.

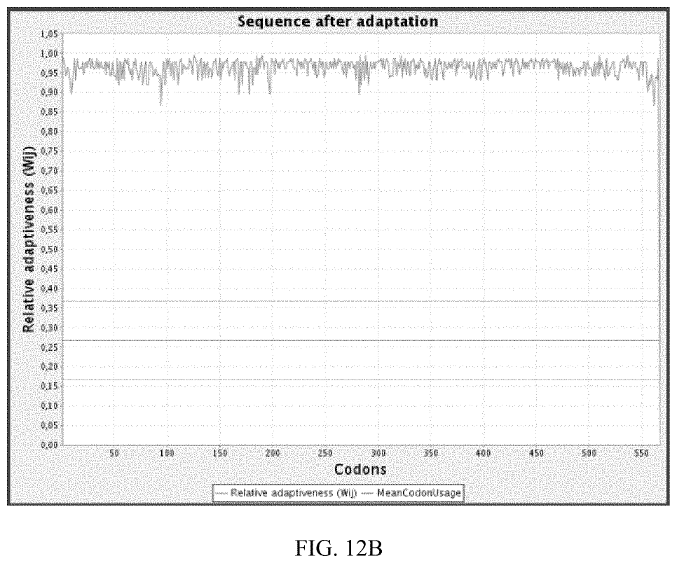

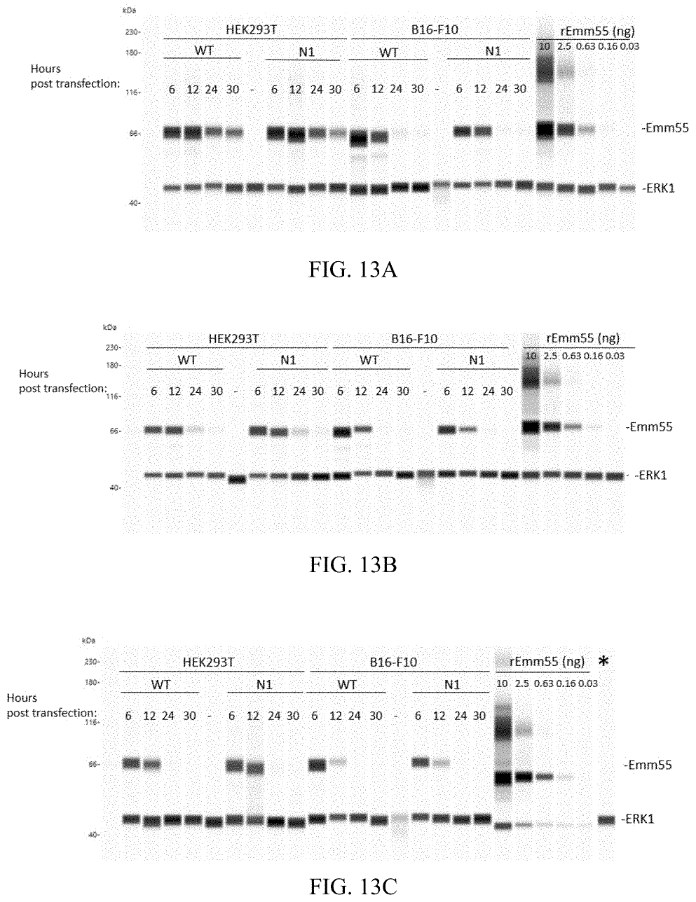

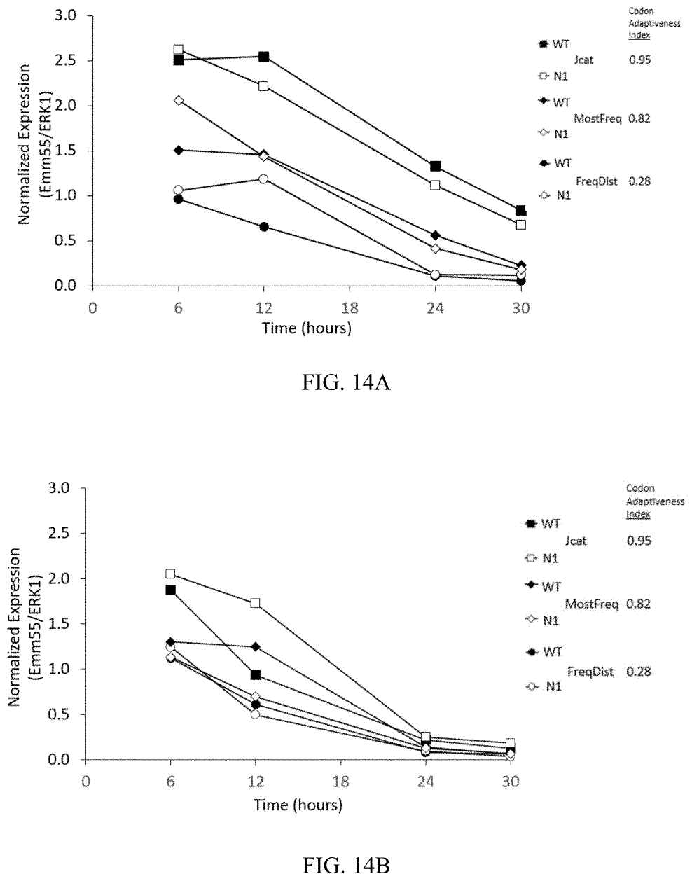

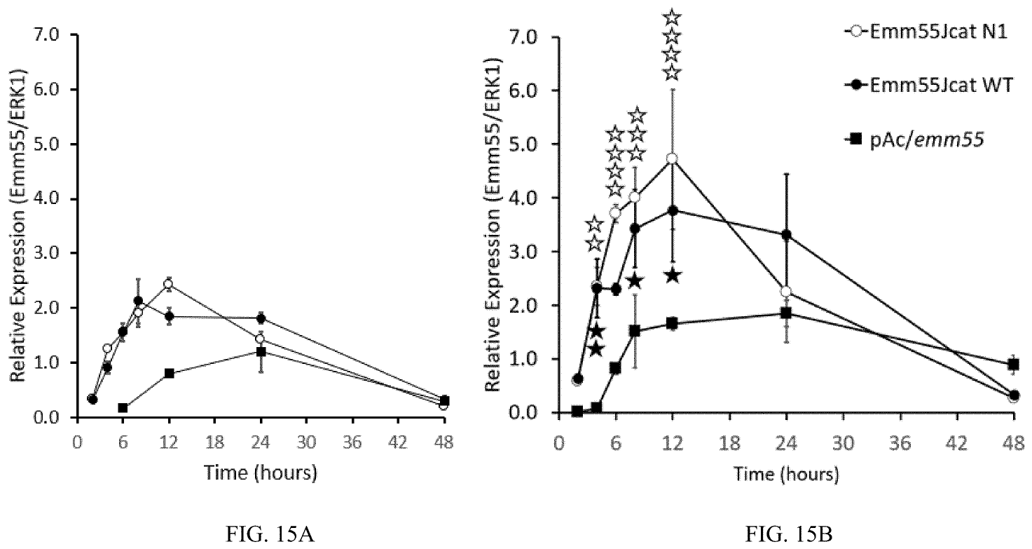

A codon adaptiveness index (CAI) was calculated using the JCat algorithm for each codon of emm55. This algorithm returns a score of 1.0 if the mRNA sequence is perfectly optimized for translation in human cells. FIG. 12 shows the relative adaptiveness of emm55 before and after optimization with JCat. A relative adaptiveness plot of emm55 is shown in FIGS. 12A and 12B.

Results showed that codon modified emm55 mRNA drives robust expression of Emm55 polypeptide following transient transfection of human HE293T cells and murine B16-F10 melanoma cells in vitro. A comparison of protein levels to levels of transfection with pAc/emm55 (the DNA) is not quantitative since a single copy of pDNA gives rise to many copies of mRNA but did show that WT and N1-modified emm55JCat expressed significantly higher levels of Emm55 in HEK293 cells between 4- and 12-hours post transfection. Peak Emm55 expression from emm55JCat mRNA was approximately 12 hours post-transfection and was detected at 48 hours.

We claim:

1. A codon optimized ribonucleic acid for expression in human cells that expresses the polynucleotide encoded by SEQ ID NO: 14 in a cell transformed with the ribonucleic acid.

2. The codon optimized ribonucleic acid of claim 1 which is selected from the group consisting of SEQ ID NO: 17, SEQ ID NO: 18 and SEQ ID NO: 19.

3. The codon optimized ribonucleic acid of claim 1 wherein the transformed cell is a cancer cell.

4. The codon optimized ribonucleic acid of claim 3 wherein the transformed cancer cell induces an immunogenic response when introduced into a cancer patient.

5. The codon optimized ribonucleic acid of claim 3 wherein the cancer cell is selected from a carcinoma, sarcoma, myeloma, or lymphoma cell or mixtures of two or more of said cells.

6. The codon optimized ribonucleic acid of claim 1 which expresses an immunogenic polynucleotide when introduced into a tumor or tumor draining lymph node in vivo.

7. The codon optimized ribonucleic acid of claim 6 wherein the tumor or tumor draining lymph node comprises carcinoma, sarcoma, myeloma, or lymphoma cells.

8. A synthetic ribonucleic acid comprising a proximal design element which increases expression of a polypeptide having the sequence of SEQ ID NO: 14 in a mammalian cell transformed with the ribonucleic acid.

9. The synthetic ribonucleic acid of claim 8 wherein the proximal design element is the translation initiation sequence.

10. The synthetic ribonucleic acid of claim 9 selected from the group consisting of SEQ ID NO: 13, SEQ ID NO: 15 or SEQ ID NO: 16.

11. The synthetic ribonucleic acid of claim 8 which expresses the polypeptide in a cancer cell transformed with the nucleic acid.

12. The synthetic ribonucleic acid of claim 11 wherein the cancer cell is a carcinoma, sarcoma, myeloma, or lymphoma cell.

13. The synthetic ribonucleic acid of claim 12 wherein the cancer cell is obtained from a cancer patient.

14. The synthetic ribonucleic acid of claim 13 wherein the cancer cell is transformed with said ribonucleic acid in vitro.

15. The synthetic ribonucleic acid of claim 14 wherein the transformed cancer cell is administered to a cancer patient.

16. The ribonucleic acid of claim 12 wherein the cancer cell is a cell from a cancer patient.

17. The synthetic ribonucleic acid of claim 10 wherein the ribonucleic acid is introduced directly into a tumor or tumor draining lymph node of a cancer patient.

18. The synthetic ribonucleic acid of claim 17 wherein the ribonucleic acid comprises a nuclease free aqueous composition.


SEQUENCE LISTING (19 sequences)

SEQ ID NO: 1 - RNA, 1854 nt, Artificial Sequence
  Theoretical emm55 mRNA sequence
  misc_feature (1)..(2): 5′ ARCA methylguanosine cap
  misc_feature (3)..(132): 5′ untranslated region
  misc_feature (133)..(135): start codon
  misc_feature (133)..(1804): emm55 gene
  misc_feature (1806)..(1854): Poly(A) tail, minimum of 50 adenosines
gggagaagaucuuugucgauccuaccauccacucgacacacccgccagcggccgcugccaagcuuccgagcucaagcuucgaauucugcagucgacgguaccgcgggcccgggauccauaaggagcauaaaaauggcuaaaaauaccacgaauagacacuauucgcuuagaaaauuaaaaacaggaacggcuucaguagcaguagcuuugacuguuuuagggacaggacugguagcagggcagacaguaaaagcaagccaaacagaaccaucucagaccaauaacagauuauaucaagaaagacaacguuuacaggauuuaaaaaguaaguuucaagaccugaaaaaucguucagagggauacauucagcaauacuacgacgaagaaaagaacaguggaaguaacucuaacugguacgcaaccuacuuaaaagaauuaaaugacgaauuugaacaagcuuauaaugaacuuaguggugaugguguaaaaaaauuagcugcaaguuugauggaagaaagagucgcuuuaagagacgaaaucgaucagauuaagaaaauaucagaagaauuaaaaaauaagcugagagcaaaagaagaagaauuaaaaaauaaaaaagaggaacgugagcuugagcaugcugccuaugcagcagaugcaaagaaacaugaagaauaugucaaauccaugucucucguacuaauggauaaagaagaggagcgucauaaacuagagcaaucauuagacacggcuaaagcugagcuuguuaaaaaagagcaagaguuacaguuagucaaaggcaaucuagaucaaaaagaaaaagaacuagaaaaugaagagcuagcgaaagaaagugcuauuagugauuugacugagcagauuacugcuaagaaggcugaaguagaaaaauuaacucaagauuuagcugcuaagucugcugaaauucaggaaaaagaagcugaaaaagaucgccaacagcauauguacgaagcguuuaugagccaguacaaagaaaaaguugagaaacaagagcaagagcuugcuaagcuaaaacaacuugaaaccaucaacaacaaucuauuagguaaugcuaaggauaugauagcuaaguugucugcugaaaaugaacaauuagcaagcgacaaagcaaaacuugaagaacaaaacaagauuucagaagcgagccguaaaggucuucgucgugacuuggacgcaucacgugaagcuaagaaacaaguugaaaaagauuuagcaaacuugacugcugaacuugauaagguuaaagaagauaaacaaauuucagacgcaagccguaaaggucuucgucgugacuuggacgcaucacgugaagcuaagaaacaaguugaaaaagcuuuagaagaagcaaacagcaaauuagcggcucuugaaaaacuuaacaaagagcuugaagaaagcaagaaauuaacagaaaaagaaaaagcugagcuacaagcgaaacuugaagcagaagcaaaagcacucaaagaacaauuagcgaaacaagcugaagaacuugcaaaacuaagagcuggaaaagcaucagacucacaaaccccugaugcaaaaccaggaaacaaaguuguuccagguacaggucaagcaccacaagcaggcacaaaaccuaaccaaaacaaagcaccaaugaaggaaacuaagagacaguuaccaucaacaggugaagcagcuaauccauucuuuacagcggcagcccuuacuguuauggcaacagcuggaguagcagcaguuguaaaacgcaaagaagaaaacgaagcugaauucugcagauaaaaaaaaaaaaaaaaaaaaaaaaaaaaaaaaaaaaaaaaaaaaaaaaaa

SEQ ID NO: 2 - protein, 557 aa, Artificial Sequence
  Emm55 Protein Sequence
MAKNTTNRHYSLRKLKTGTASVAVALTVLGTGLVAGQTVKASQTEPSQTNNRLYQERQRLQDLKSKFQDLKNRSEGYIQQYYDEEKNSGSNSNWYATYLKELNDEFEQAYNELSGDGVKKLAASLMEERVALRDEIDQIKKISEELKNKLRAKEEELKNKKEERELEHAAYAADAKKHEEYVKSMSLVLMDKEEERHKLEQSLDTAKAELVKKEQELQLVKGNLDQKEKELENEELAKESAISDLTEQITAKKAEVEKLTQDLAAKSAEIQEKEAEKDRQQHMYEAFMSQYKEKVEKQEQELAKLKQLETINNNLLGNAKDMIAKLSAENEQLASDKAKLEEQNKISEASRKGLRRDLDASREAKKQVEKDLANLTAELDKVKEDKQISDASRKGLRRDLDASREAKKQVEKALEEANSKLAALEKLNKELEESKKLTEKEKAELQAKLEAEAKALKEQLAKQAEELAKLRAGKASDSQTPDAKPGNKVVPGTGQAPQAGTKPNQNKAPMKETKRQLPSTGEAANPFFTAAALTVMATAGVAAVVKRKEENEAEFCR

SEQ ID NO: 3 - RNA, 179 nt, Artificial Sequence
  Theoretical untranslated mRNA sequence of SEQ ID NO 1
  misc_feature (1)..(2): 5′ ARCA methylguanosine cap
  misc_feature (3)..(132): 5′ untranslated region
  misc_feature (133)..(135): start codon
  misc_feature (136)..(179): Poly(A) tail, minimum of 50 adenosines
gggagaagaucuuugucgauccuaccauccacucgacacacccgccagcggccgcugccaagcuuccgagcucaagcuucgaauucugcagucgacgguaccgcgggcccgggauccauaaggagcauaaaaaugaaaaaaaaaaaaaaaaaaaaaaaaaaaaaaaaaaaaaaaaaaaa

SEQ ID NO: 4 - DNA, 11 bp, Artificial Sequence
  Degenerate DNA sequence of an optimal translation initiation sequence
  misc_feature (1)..(1): R is A or G
  misc_feature (2)..(2): Y is C or T
  misc_feature (3)..(3): M is A or C
  misc_feature (4)..(4): R is A or G
  misc_feature (5)..(5): M is A or C
  misc_feature (6)..(6): V is A , C or G
rymrmvatggc

SEQ ID NO: 5 - DNA, 11 bp, Artificial Sequence
  DNA sequence of an optimal translation initiation sequence
atagccatggc

SEQ ID NO: 6 - DNA, 6 bp, Artificial Sequence
  DNA sequence containing 2 stop codons
taatga

SEQ ID NO: 7 - DNA, 32 bp, Artificial Sequence
  DNA sequence of the restriction endonuclease recognition sites for SacI, NotI, BglII, EcoRV and SpeI
gagctcgcggccgcagatctgatatcactagt

SEQ ID NO: 8 - DNA, 18 bp, Artificial Sequence
  DNA sequence of the T7 RNA polymerase promoter
taatacgactcactatag

SEQ ID NO: 9 - RNA, 50 nt, Xenopus laevis
  misc_feature (1)..(50): 5′ untranslated region
aagcuucuuguucuuuuugcagaagcucagaauaaacgcucaacuuuggc

SEQ ID NO: 10 - RNA, 74 nt, Xenopus laevis
  misc_feature (1)..(74): 3′ untranslated region
cuuuuugaugccauugccgacgcccuuggcaaggguuaccacuaaaccagccucaagaacacccgaauggaguc

SEQ ID NO: 11 - DNA, 18 bp, Artificial Sequence
  DNA sequence of the restriction endonuclease recognition sites for BamHI, EcoRI and XbaI
ggatccgaattctctaga

SEQ ID NO: 12 - DNA, 192 bp, Artificial Sequence
  DNA sequence of the synthetic double stranded DNA molecule used to prepare pT7XLUTR
taatacgactcactatagaagcttcttgttctttttgcagaagctcagaataaacgctcaactttggcgagctcgcggccgcagatctgatatcactagtctttttgatgccattgccgacgcccttggcaagggttaccactaaaccagcctcaagaacacccgaatggagtcggatccgaattctctaga

SEQ ID NO: 13 - RNA, 1851 nt, Artificial Sequence
  Synthetic
gggagaagaucuuugucgauccuaccauccacucgacacacccgccagcggccgcugccaagcuuccgagcucaagcuucgaauucugcagucgacgguaccgcgggcccgggauccauaaggagcauaaaaauagccauggcuaaaaauaccacgaauagacacuauucgcuuagaaaauuaaaaacaggaacggcuucaguagcaguagcuuugacuguuuuagggacaggacugguagcagggcagacaguaaaagcaagccaaacagaaccaucucagaccaauaacagauuauaucaagaaagacaacguuuacaggauuuaaaaaguaaguuucaagaccugaaaaaucguucagagggauacauucagcaauacuacgacgaagaaaagaacaguggaaguaacucuaacugguacgcaaccuacuuaaaagaauuaaaugacgaauuugaacaagcuuauaaugaacuuaguggugaugguguaaaaaaauuagcugcaaguuugauggaagaaagagucgcuuuaagagacgaaaucgaucagauuaagaaaauaucagaagaauuaaaaaauaagcugagagcaaaagaagaagaauuaaaaaauaaaaaagaggaacgugagcuugagcaugcugccuaugcagcagaugcaaagaaacaugaagaauaugucaaauccaugucucucguacuaauggauaaagaagaggagcgucauaaacuagagcaaucauuagacacggcuaaagcugagcuuguuaaaaaagagcaagaguuacaguuagucaaaggcaaucuagaucaaaaagaaaaagaacuagaaaaugaagagcuagcgaaagaaagugcuauuagugauuugacugagcagauuacugcuaagaaggcugaaguagaaaaauuaacucaagauuuagcugcuaagucugcugaaauucaggaaaaagaagcugaaaaagaucgccaacagcauauguacgaagcguuuaugagccaguacaaagaaaaaguugagaaacaagagcaagagcuugcuaagcuaaaacaacuugaaaccaucaacaacaaucuauuagguaaugcuaaggauaugauagcuaaguugucugcugaaaaugaacaauuagcaagcgacaaagcaaaacuugaagaacaaaacaagauuucagaagcgagccguaaaggucuucgucgugacuuggacgcaucacgugaagcuaagaaacaaguugaaaaagauuuagcaaacuugacugcugaacuugauaagguuaaagaagauaaacaaauuucagacgcaagccguaaaggucuucgucgugacuuggacgcaucacgugaagcuaagaaacaaguugaaaaagcuuuagaagaagcaaacagcaaauuagcggcucuugaaaaacuuaacaaagagcuugaagaaagcaagaaauuaacagaaaaagaaaaagcugagcuacaagcgaaacuugaagcagaagcaaaagcacucaaagaacaauuagcgaaacaagcugaagaacuugcaaaacuaagagcuggaaaagcaucagacucacaaaccccugaugcaaaaccaggaaacaaaguuguuccagguacaggucaagcaccacaagcaggcacaaaaccuaaccaaaacaaagcaccaaugaaggaaacuaagagacaguuaccaucaacaggugaagcagcuaauccauucuuuacagcggcagcccuuacuguuauggcaacagcuggaguagcagcaguuguaaaacgcaaagaagaaaacgaagcugaauucugcagauauccaucacacuggcggccgcgacucuaguaaugacucgag

SEQ ID NO: 14 - protein, 566 aa, Artificial Sequence
  Synthetic
MAKNTTNRHYSLRKLKTGTASVAVALTVLGTGLVAGQTVKASQTEPSQTNNRLYQERQRLQDLKSKFQDLKNRSEGYIQQYYDEEKNSGSNSNWYATYLKELNDEFEQAYNELSGDGVKKLAASLMEERVALRDEIDQIKKISEELKNKLRAKEEELKNKKEERELEHAAYAADAKKHEEYVKSMSLVLMDKEEERHKLEQSLDTAKAELVKKEQELQLVKGNLDQKEKELENEELAKESAISDLTEQITAKKAEVEKLTQDLAAKSAEIQEKEAEKDRQQHMYEAFMSQYKEKVEKQEQELAKLKQLETINNNLLGNAKDMIAKLSAENEQLASDKAKLEEQNKISEASRKGLRRDLDASREAKKQVEKDLANLTAELDKVKEDKQISDASRKGLRRDLDASREAKKQVEKALEEANSKLAALEKLNKELEESKKLTEKEKAELQAKLEAEAKALKEQLAKQAEELAKLRAGKASDSQTPDAKPGNKVVPGTGQAPQAGTKPNQNKAPMKETKRQLPSTGEAANPFFTAAALTVMATAGVAAVVKRKEENEAEFCRYPSHWRPRL

SEQ ID NO: 15 - RNA, 1856 nt, Artificial Sequence
  Synthetic
gaagcuucuuguucuuuuugcagaagcucagaauaaacgcucaacuuuggcgagcucauagccauggcuaaaaauaccacgaauagacacuauucgcuuagaaaauuaaaaacaggaacggcuucaguagcaguagcuuugacuguuuuagggacaggacugguagcagggcagacaguaaaagcaagccaaacagaaccaucucagaccaauaacagauuauaucaagaaagacaacguuuacaggauuuaaaaaguaaguuucaagaccugaaaaaucguucagagggauacauucagcaauacuacgacgaagaaaagaacaguggaaguaacucuaacugguacgcaaccuacuuaaaagaauuaaaugacgaauuugaacaagcuuauaaugaacuuaguggugaugguguaaaaaaauuagcugcaaguuugauggaagaaagagucgcuuuaagagacgaaaucgaucagauuaagaaaauaucagaagaauuaaaaaauaagcugagagcaaaagaagaagaauuaaaaaauaaaaaagaggaacgugagcuugagcaugcugccuaugcagcagaugcaaagaaacaugaagaauaugucaaauccaugucucucguacuaauggauaaagaagaggagcgucauaaacuagagcaaucauuagacacggcuaaagcugagcuuguuaaaaaagagcaagaguuacaguuagucaaaggcaaucuagaucaaaaagaaaaagaacuagaaaaugaagagcuagcgaaagaaagugcuauuagugauuugacugagcagauuacugcuaagaaggcugaaguagaaaaauuaacucaagauuuagcugcuaagucugcugaaauucaggaaaaagaagcugaaaaagaucgccaacagcauauguacgaagcguuuaugagccaguacaaagaaaaaguugagaaacaagagcaagagcuugcuaagcuaaaacaacuugaaaccaucaacaacaaucuauuagguaaugcuaaggauaugauagcuaaguugucugcugaaaaugaacaauuagcaagcgacaaagcaaaacuugaagaacaaaacaagauuucagaagcgagccguaaaggucuucgucgugacuuggacgcaucacgugaagcuaagaaacaaguugaaaaagauuuagcaaacuugacugcugaacuugauaagguuaaagaagauaaacaaauuucagacgcaagccguaaaggucuucgucgugacuuggacgcaucacgugaagcuaagaaacaaguugaaaaagcuuuagaagaagcaaacagcaaauuagcggcucuugaaaaacuuaacaaagagcuugaagaaagcaagaaauuaacagaaaaagaaaaagcugagcuacaagcgaaacuugaagcagaagcaaaagcacucaaagaacaauuagcgaaacaagcugaagaacuugcaaaacuaagagcuggaaaagcaucagacucacaaaccccugaugcaaaaccaggaaacaaaguuguuccagguacaggucaagcaccacaagcaggcacaaaaccuaaccaaaacaaagcaccaaugaaggaaacuaagagacaguuaccaucaacaggugaagcagcuaauccauucuuuacagcggcagcccuuacuguuauggcaacagcuggaguagcagcaguuguaaaacgcaaagaagaaaacgaagcugaauucugcagauauccaucacacuggcggccgcgacucuaguaaugaacuagucuuuuugaugccauugccgacgcccuuggcaaggguuaccacuaaaccagccucaagaacacccgaauggagucggaucc

SEQ ID NO: 16 - RNA, 1720 nt, Artificial Sequence
  Synthetic
gauagccauggcuaaaaauaccacgaauagacacuauucgcuuagaaaauuaaaaacaggaacggcuucaguagcaguagcuuugacuguuuuagggacaggacugguagcagggcagacaguaaaagcaagccaaacagaaccaucucagaccaauaacagauuauaucaagaaagacaacguuuacaggauuuaaaaaguaaguuucaagaccugaaaaaucguucagagggauacauucagcaauacuacgacgaagaaaagaacaguggaaguaacucuaacugguacgcaaccuacuuaaaagaauuaaaugacgaauuugaacaagcuuauaaugaacuuaguggugaugguguaaaaaaauuagcugcaaguuugauggaagaaagagucgcuuuaagagacgaaaucgaucagauuaagaaaauaucagaagaauuaaaaaauaagcugagagcaaaagaagaagaauuaaaaaauaaaaaagaggaacgugagcuugagcaugcugccuaugcagcagaugcaaagaaacaugaagaauaugucaaauccaugucucucguacuaauggauaaagaagaggagcgucauaaacuagagcaaucauuagacacggcuaaagcugagcuuguuaaaaaagagcaagaguuacaguuagucaaaggcaaucuagaucaaaaagaaaaagaacuagaaaaugaagagcuagcgaaagaaagugcuauuagugauuugacugagcagauuacugcuaagaaggcugaaguagaaaaauuaacucaagauuuagcugcuaagucugcugaaauucaggaaaaagaagcugaaaaagaucgccaacagcauauguacgaagcguuuaugagccaguacaaagaaaaaguugagaaacaagagcaagagcuugcuaagcuaaaacaacuugaaaccaucaacaacaaucuauuagguaaugcuaaggauaugauagcuaaguugucugcugaaaaugaacaauuagcaagcgacaaagcaaaacuugaagaacaaaacaagauuucagaagcgagccguaaaggucuucgucgugacuuggacgcaucacgugaagcuaagaaacaaguugaaaaagauuuagcaaacuugacugcugaacuugauaagguuaaagaagauaaacaaauuucagacgcaagccguaaaggucuucgucgugacuuggacgcaucacgugaagcuaagaaacaaguugaaaaagcuuuagaagaagcaaacagcaaauuagcggcucuugaaaaacuuaacaaagagcuugaagaaagcaagaaauuaacagaaaaagaaaaagcugagcuacaagcgaaacuugaagcagaagcaaaagcacucaaagaacaauuagcgaaacaagcugaagaacuugcaaaacuaagagcuggaaaagcaucagacucacaaaccccugaugcaaaaccaggaaacaaaguuguuccagguacaggucaagcaccacaagcaggcacaaaaccuaaccaaaacaaagcaccaaugaaggaaacuaagagacaguuaccaucaacaggugaagcagcuaauccauucuuuacagcggcagcccuuacuguuauggcaacagcuggaguagcagcaguuguaaaacgcaaagaagaaaacgaagcugaauucugcagauauccaucacacuggcggccgcgacucuaguaaugacucgag

SEQ ID NO: 17 - DNA, 1707 bp, Artificial Sequence
  Synthetic
gccaccatggccaagaacaccaccaaccgccactacagcctgcgcaagctcaagaccggcaccgccagcgtggccgtggccctgaccgtgctgggcaccggcctggtggccggccagaccgtgaaggccagccagaccgagcccagccagaccaacaaccgcctgtaccaggagcgccagcgcctgcaggacctgaaaagcaagttccaggacctgaagaaccgcagcgagggctacatccagcagtactacgacgaggagaagaacagcggcagcaacagcaactggtacgccacctacctgaaggagctgaacgacgagttcgagcaggcctacaacgagctgagcggcgacggcgtgaagaagctggccgccagcctgatggaggagcgcgtggccctgcgcgacgagatcgaccagatcaagaagatcagcgaggagctgaagaacaagctgcgcgccaaggaggaggagctgaagaacaagaaggaggagcgcgagctggagcacgccgcctacgccgccgacgccaagaagcacgaggagtacgtgaaaagcatgagcctggtgctgatggacaaggaggaggagcgccacaagctggagcagagcctggacaccgccaaggccgagctggtgaagaaggagcaggagctgcagctggtgaagggcaacctggaccagaaggagaaggagctggagaacgaggagctggccaaggagagcgccatcagcgacctgaccgagcagatcaccgccaagaaggccgaggtggagaagctgacccaggacctggccgccaagagcgccgagatccaggagaaggaggccgagaaggaccgccagcagcacatgtacgaggccttcatgagccagtacaaggagaaggtggagaagcaggagcaggagctggccaagctgaagcagctggagaccatcaacaacaacctgctgggcaacgccaaggacatgatcgccaagctgagcgccgagaacgagcagctggccagcgacaaggccaagctggaggagcagaacaagatcagcgaggccagccgcaagggcctgcgccgcgacctggacgccagccgcgaggccaagaagcaggtggagaaggacctggccaacctgaccgccgagctggacaaggtgaaggaggacaagcagatcagcgacgccagccgcaagggcctgcgccgcgacctggacgccagccgcgaggccaagaagcaggtggagaaggccctggaggaggccaacagcaagctggccgccctggagaagctgaacaaggagctggaggagagcaagaagctgaccgagaaggagaaggccgagctgcaggccaagctggaggccgaggccaaggccctgaaggagcagctggccaagcaggccgaggagctggccaagctgcgcgccggcaaggccagcgacagccagacccccgacgccaagcccggcaacaaggtggtgcccggcaccggccaggccccccaggccggcaccaagcccaaccagaacaaggcccccatgaaggagaccaagcgccagctgcccagcaccggcgaggccgccaaccccttcttcaccgccgccgccctgaccgtgatggccaccgccggcgtggccgccgtggtgaagcgcaaggaggagaacgaggccgagttctgccgctaccccagccactggcgcccccgcctgtaa

SEQ ID NO: 18 - DNA, 1707 bp, Artificial Sequence
  Synthetic
gccaccatggccaagaacaccaccaacagacactacagcctgagaaagctcaagaccggcaccgccagcgtggccgtggccctgaccgtgctgggcaccggcctggtggccggccagaccgtgaaggccagccagaccgagcccagccagaccaacaacagactgtaccaggagagacagagactgcaggacctgaaaagcaagttccaggacctgaagaacagaagcgagggctacatccagcagtactacgacgaggagaagaacagcggcagcaacagcaactggtacgccacctacctgaaggagctgaacgacgagttcgagcaggcctacaacgagctgagcggcgacggcgtgaagaagctggccgccagcctgatggaggagagagtggccctgagagacgagatcgaccagatcaagaagatcagcgaggagctgaagaacaagctgagagccaaggaggaggagctgaagaacaagaaggaggagagagagctggagcacgccgcctacgccgccgacgccaagaagcacgaggagtacgtgaaaagcatgagcctggtgctgatggacaaggaggaggagagacacaagctggagcagagcctggacaccgccaaggccgagctggtgaagaaggagcaggagctgcagctggtgaagggcaacctggaccagaaggagaaggagctggagaacgaggagctggccaaggagagcgccatcagcgacctgaccgagcagatcaccgccaagaaggccgaggtggagaagctgacccaggacctggccgccaagagcgccgagatccaggagaaggaggccgagaaggacagacagcagcacatgtacgaggccttcatgagccagtacaaggagaaggtggagaagcaggagcaggagctggccaagctgaagcagctggagaccatcaacaacaacctgctgggcaacgccaaggacatgatcgccaagctgagcgccgagaacgagcagctggccagcgacaaggccaagctggaggagcagaacaagatcagcgaggccagcagaaagggcctgagaagagacctggacgccagcagagaggccaagaagcaggtggagaaggacctggccaacctgaccgccgagctggacaaggtgaaggaggacaagcagatcagcgacgccagcagaaagggcctgagaagagacctggacgccagcagagaggccaagaagcaggtggagaaggccctggaggaggccaacagcaagctggccgccctggagaagctgaacaaggagctggaggagagcaagaagctgaccgagaaggagaaggccgagctgcaggccaagctggaggccgaggccaaggccctgaaggagcagctggccaagcaggccgaggagctggccaagctgagagccggcaaggccagcgacagccagacccccgacgccaagcccggcaacaaggtggtgcccggcaccggccaggccccccaggccggcaccaagcccaaccagaacaaggcccccatgaaggagaccaagagacagctgcccagcaccggcgaggccgccaaccccttcttcaccgccgccgccctgaccgtgatggccaccgccggcgtggccgccgtggtgaagagaaaggaggagaacgaggccgagttctgcagataccccagccactggagacccagactgtaa

SEQ ID NO: 19 - DNA, 1707 bp, Artificial Sequence
  Synthetic
gccaccatggcaaagaataccacgaatagacattattcattaagaaagctcaagaccggcacggcatccgtggcagtggctttgacagtgctgggcacaggcctcgtggccggccagacagttaaggccagccagacagagccatcacaaacgaataaccggctgtaccaggagcggcaaagactgcaggacttaaagtctaaatttcaggatcttaaaaaccggagtgaaggttatatccagcagtattatgacgaggagaagaactccgggagcaatagcaactggtacgcaacctatctgaaggaacttaatgacgagtttgaacaggcttataatgaactgtccggtgatggagtaaagaaattggccgctagtctcatggaagagcgagtggctttgcgggatgagattgaccagattaagaaaatctccgaagagctgaaaaataaacttagggctaaggaggaggagctcaaaaataaaaaggaagaaagagaactggagcacgccgcgtacgccgctgatgctaaaaaacatgaggagtatgtcaagagcatgtccctggtcctgatggacaaggaggaagagcggcacaagctcgagcagtcactggataccgcgaaagcggaactggtgaagaaagaacaagaactccaactagtgaaaggcaatttagaccagaaggagaaggaactagaaaatgaagagctggcgaaagagtctgccatatcagacttgacagagcagatcactgctaagaaggccgaagtggaaaagttgacccaagacttagcagctaaatcggccgagattcaagagaaggaggctgagaaggaccgccagcagcacatgtacgaagcttttatgtcacagtacaaagagaaagtcgagaaacaggagcaggaactggccaaactgaagcagctggagacaataaataataatctactgggcaacgctaaggatatgatcgctaaactgagcgctgaaaacgagcagttagcctccgataaagcgaaattggaagagcaaaacaagatctccgaggccagcaggaaaggccttagaagggacctagacgcctcgagagaggccaaaaagcaggtcgagaaggacctagcaaaccttaccgccgagctggacaaagttaaggaggataagcagatcagcgacgccagtagaaaaggcttaaggagggacttggacgctagtagagaggccaaaaagcaggtggaaaaagcgttagaggaggcgaattccaaattagcggcattggagaagctgaacaaggagctggaggagtcgaagaagttaacggagaaggaaaaagcagagttgcaagcaaagttggaggccgaggcaaaggcattaaaagaacagctcgctaaacaggcggaggaactggcaaaactgagagcaggcaaggcctccgacagtcagacgcctgatgcaaagcctgggaataaggtggtgcctggcacggggcaggccccccaggctggcacgaagccaaatcaaaacaaggctccaatgaaggagactaagcgccagctgcctagtaccggagaggccgccaatccttttttcaccgccgccgcccttactgttatggccactgctggagttgcggctgtggtgaaacggaaagaggaaaacgaagccgaattttgccggtaccctagtcattggagaccaagattgtaa